SEQUENCE LISTING (118 sequences)

SEQ ID NO: 1 - protein, 439 aa, Homo sapiens
QVQLQQSGAELVKPGASVKLSCKASGYTFTSYDINWVRQRPEQGLEWIGWIFPGDDTTNYNENFKGKATLTADKSSSTAYMQLTRLTSEDSAVFFCARSVGFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 2 - protein, 19 aa, Homo sapiens
MGWSCVFLFLLSVTAGVHS

SEQ ID NO: 3 - protein, 115 aa, Homo sapiens
QVQLQQSGAELVKPGASVKLSCKASGYTFTSYDINWVRQRPEQGLEWIGWIFPGDDTTNYNENFKGKATLTADKSSSTAYMQLTRLTSEDSAVFFCARSVGFDYWGQGTTLTVSS

SEQ ID NO: 4 - protein, 5 aa, Homo sapiens
SYDIN

SEQ ID NO: 5 - protein, 17 aa, Homo sapiens
WIFPGDDTTNYNENFKG

SEQ ID NO: 6 - protein, 6 aa, Homo sapiens
SVGFDY

SEQ ID NO: 7 - protein, 30 aa, Homo sapiens
QVQLQQSGAELVKPGASVKLSCKASGYTFT

SEQ ID NO: 8 - protein, 14 aa, Homo sapiens
WVRQRPEQGLEWIG

SEQ ID NO: 9 - protein, 32 aa, Homo sapiens
KATLTADKSSSTAYMQLTRLTSEDSAVFFCAR

SEQ ID NO: 10 - protein, 11 aa, Homo sapiens
WGQGTTLTVSS

SEQ ID NO: 11 - protein, 212 aa, Homo sapiens
QIVLIQSPAIMSASPGEKVTMTCSASSSISYMYWYQQKPGTSPKRWIFETSKLASGVPARFSGSGSGTSYSLTISSMEAEDAATYYCHQRSSYPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 12 - protein, 22 aa, Homo sapiens
MDFQVRIFSFLLISASVILSRG

SEQ ID NO: 13 - protein, 105 aa, Homo sapiens
QIVLIQSPAIMSASPGEKVTMTCSASSSISYMYWYQQKPGTSPKRWIFETSKLASGVPARFSGSGSGTSYSLTISSMEAEDAATYYCHQRSSYPTFGGGTKLEIK

SEQ ID NO: 14 - protein, 10 aa, Homo sapiens
SASSSISYMY

SEQ ID NO: 15 - protein, 7 aa, Homo sapiens
ETSKLAS

SEQ ID NO: 16 - protein, 8 aa, Homo sapiens
HQRSSYPT

SEQ ID NO: 17 - protein, 23 aa, Homo sapiens
QIVLIQSPAIMSASPGEKVTMTC

SEQ ID NO: 18 - protein, 15 aa, Homo sapiens
WYQQKPGTSPKRWIF

SEQ ID NO: 19 - protein, 32 aa, Homo sapiens
GVPARFSGSGSGTSYSLTISSMEAEDAATYYC

SEQ ID NO: 20 - protein, 10 aa, Homo sapiens
FGGGTKLEIK

SEQ ID NO: 21 - DNA, 1317 bp, Homo sapiens
  source 1..1317: mol_type = other DNA
caggttcagctgcagcagtctggagctgaactggtaaagcctggggcttcagtgaagttgtcctgcaaggcttctggctacaccttcacaagttatgatataaactgggtgaggcagaggcctgaacagggacttgagtggattggatggatttttcctggagatgatactactaactacaatgagaatttcaagggcaaggccacactgactgcagacaaatcctccagcacagcctacatgcagctcaccaggctgacatctgaggactctgctgtctttttctgtgcaagatcggtgggctttgactactggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 22 - DNA, 57 bp, Homo sapiens
  source 1..57: mol_type = other DNA
atgggatggagctgtgtctttctcttcctcctgtcagtaactgcaggtgtccactcc

SEQ ID NO: 23 - DNA, 345 bp, Homo sapiens
  source 1..345: mol_type = other DNA
caggttcagctgcagcagtctggagctgaactggtaaagcctggggcttcagtgaagttgtcctgcaaggcttctggctacaccttcacaagttatgatataaactgggtgaggcagaggcctgaacagggacttgagtggattggatggatttttcctggagatgatactactaactacaatgagaatttcaagggcaaggccacactgactgcagacaaatcctccagcacagcctacatgcagctcaccaggctgacatctgaggactctgctgtctttttctgtgcaagatcggtgggctttgactactggggccaaggcaccactctcacagtctcctca

SEQ ID NO: 24 - DNA, 636 bp, Homo sapiens
  source 1..636: mol_type = other DNA
caaattgttctcatccagtctccagcaatcatgtctgcatctccaggggagaaggtcaccatgacctgcagtgccagttcaagtataagttacatgtactggtaccagcagaagccaggcacctcccccaaaagatggatttttgaaacatccaaactggcttctggagtccctgctcgcttcagtggcagtgggtctgggacctcttattctctcacaatcagcagcatggaggctgaagatgctgccacttattactgccatcagcggagtagttaccccacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 25 - DNA, 66 bp, Homo sapiens
  source 1..66: mol_type = other DNA
atggattttcaggtgcggattttcagcttcctgctaatcagtgcctcagtcatactgtccagagga

SEQ ID NO: 26 - DNA, 315 bp, Homo sapiens
  source 1..315: mol_type = other DNA
caaattgttctcatccagtctccagcaatcatgtctgcatctccaggggagaaggtcaccatgacctgcagtgccagttcaagtataagttacatgtactggtaccagcagaagccaggcacctcccccaaaagatggatttttgaaacatccaaactggcttctggagtccctgctcgcttcagtggcagtgggtctgggacctcttattctctcacaatcagcagcatggaggctgaagatgctgccacttattactgccatcagcggagtagttaccccacgttcggaggggggaccaagctggaaataaaa

SEQ ID NO: 27 - protein, 5 aa, Homo sapiens
DKTHT

SEQ ID NO: 28 - protein, 4 aa, Homo sapiens
CPPC

SEQ ID NO: 29 - protein, 15 aa, Homo sapiens
CPEPKSCDTPPPCPR

SEQ ID NO: 30 - protein, 12 aa, Homo sapiens
ELKTPLGDTTHT

SEQ ID NO: 31 - protein, 10 aa, Homo sapiens
KSCDKTHTCP

SEQ ID NO: 32 - protein, 7 aa, Homo sapiens
KCCVDCP

SEQ ID NO: 33 - protein, 7 aa, Homo sapiens
KYGPPCP

SEQ ID NO: 34 - protein, 15 aa, Homo sapiens
EPKSCDKTHTCPPCP

SEQ ID NO: 35 - protein, 17 aa, Homo sapiens
ELKTPLGDTTHTCPRCP

SEQ ID NO: 36 - protein, 12 aa, Homo sapiens
SPNMVPHAHHAQ

SEQ ID NO: 37 - protein, 12 aa, Homo sapiens
ESKYGPPCPPCP

SEQ ID NO: 38 - protein, 15 aa, Homo sapiens
EPKSCDKTYTCPPCP

SEQ ID NO: 39 - protein, 45 aa, Homo sapiens
TTTPAPRPPTPAPTIASQPLSLRPEACRPAAGGAVHTRGLDFACD

SEQ ID NO: 40 - protein, 110 aa, Homo sapiens
APEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAK

SEQ ID NO: 41 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 27 aa, Homo sapiens
FWVLVVVGGVLACYSLLVTVAFIIFWV

SEQ ID NO: 43 - protein, 23 aa, Homo sapiens
LGLLVAGVLVLLVSLGVAIHLCC

SEQ ID NO: 44 - protein, 25 aa, Homo sapiens
ALIVLGGVAGLLLFIGLGIFFCVRC

SEQ ID NO: 45 - protein, 23 aa, Homo sapiens
LCYLLDGILFIYGVILTALFLRV

SEQ ID NO: 46 - protein, 26 aa, Homo sapiens
WVLVVVGGVLACYSLLVTVAFIIFWV

SEQ ID NO: 47 - protein, 26 aa, Homo sapiens
VAAILGLGLVLGLLGPLAILLALYLL

SEQ ID NO: 48 - protein, 24 aa, Homo sapiens
ALPAALAVISFLLGLGLGVACVLA

SEQ ID NO: 49 - protein, 113 aa, Homo sapiens
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 50 - protein, 112 aa, Homo sapiens
MGGLEPCSRLLLLPLLLAVSGLRPVQAQAQSDCSCSTVSPGVLAGIVMGDLVLTVLIALAVYFLGRLVPRGRGAAEATRKQRITETESPYQELQGQRSDVYSDLNTQRPYYK

SEQ ID NO: 51 - protein, 102 aa, Homo sapiens
MGGLEPCSRLLLLPLLLAVSDCSCSTVSPGVLAGIVMGDLVLTVLIALAVYFLGRLVPRGRGAAEAATRKQRITETESPYQELQGQRSDVYSDLNTQRPYYK

SEQ ID NO: 52 - protein, 101 aa, Homo sapiens
MGGLEPCSRLLLLPLLLAVSDCSCSTVSPGVLAGIVMGDLVLTVLIALAVYFLGRLVPRGRGAAEATRKQRITETESPYQELQGQRSDVYSDLNTQRPYYK

SEQ ID NO: 53 - protein, 86 aa, Homo sapiens
MIPAVVLLLLLLVEQAAALGEPQLCYILDAILFLYGIVLTLLYCRLKIQVRKAAITSYEKSDGVYTGLSTRNQETYETLKHEKPPQ

SEQ ID NO: 54 - protein, 21 aa, Homo sapiens
DGVYTGLSTRNQETYETLKHE

SEQ ID NO: 55 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 56 - protein, 127 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRTADTQALLRNDQVYQPLRDRDDAQYSHLGGNWARNK

SEQ ID NO: 57 - protein, 21 aa, Homo sapiens
DQVYQPLRDRDDAQYSHLGGN

SEQ ID NO: 58 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 59 - protein, 21 aa, Homo sapiens
NPDYEPIRKGQRDLYSGLNQR

SEQ ID NO: 60 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 61 - protein, 21 aa, Homo sapiens
DQLYQPLKDREDDQYSHLQGN

SEQ ID NO: 62 - protein, 163 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 64 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 21 aa, Homo sapiens
NQLYNELNLGRREEYDVLDKR

SEQ ID NO: 66 - protein, 22 aa, Homo sapiens
EGLYNELQKDKMAEAYSEIGMK

SEQ ID NO: 67 - protein, 21 aa, Homo sapiens
DGLYQGLSTATKDTYDALHMQ

SEQ ID NO: 68 - protein, 226 aa, Homo sapiens
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 69 - protein, 188 aa, Homo sapiens
MPGGPGVLQALPATIFLLFLLSAVYLGPGCQALWMHKVPASLMVSLGEDAHFQCPHNSSNNANVTWWRVLHGNYTWPPEFLGPGEDPNEPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 70 - protein, 21 aa, Homo sapiens
ENLYEGLNLDDCSMYEDISRG

SEQ ID NO: 71 - protein, 68 aa, Homo sapiens
FWVLVVVGGVLACYSLLVTVAFIIFWVRSKRSRLLHSDYMNMTPRRPGPTRKHYQPYAPPRDFAAYRS

SEQ ID NO: 72 - protein, 42 aa, Homo sapiens
KRGRKKLLYIFKQPFMRPVQTTQEEDGCSCRFPEEEEGGCEL

SEQ ID NO: 73 - protein, 44 aa, Homo sapiens
FWVRSKRSRLLHSDYMNMTPRRPGPTRKHYQPYAPPRDFAAYRS

SEQ ID NO: 74 - protein, 35 aa, Homo sapiens
TKKKYSSSVHDPNGEYMFMRAVNTAKKSRLTDVTL

SEQ ID NO: 75 - protein, 37 aa, Homo sapiens
RRDQRLPPDAHKPPGGGSFRTPIQEEQADAHSTLAKI

SEQ ID NO: 76 - protein, 114 aa, Homo sapiens
CCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 77 - protein, 49 aa, Homo sapiens
HQRRKYRSNKGESPVEPAEPCRYSCPREEEGSTIPIQEDYRKPEPACSP

SEQ ID NO: 78 - protein, 187 aa, Homo sapiens
RRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 79 - protein, 54 aa, Homo sapiens
HIWQLRSQCMWPRETQLLLEVPPSTEDARSCQFPEEERGERSAEEKGRLGDLWV

SEQ ID NO: 80 - protein, 60 aa, Homo sapiens
CVKRRKPRGDVVKVIVSVQRKRQEAEGEATVIEALQAPPDVTTVAVEETIPSFTGRSPNH

SEQ ID NO: 81 - protein, 41 aa, Homo sapiens
RSKRSRGGHSDYMNMTPRRPGPTRKHYQPYAPPRDFAAYRS

SEQ ID NO: 82 - protein, 9 aa, Homo sapiens
YPYDVPDYA

SEQ ID NO: 83 - protein, 8 aa, Homo sapiens
DYKDDDDK

SEQ ID NO: 84 - protein, 10 aa, Homo sapiens
EQKLISEEDL

SEQ ID NO: 85 - protein, 4 aa, Homo sapiens
GGSG

SEQ ID NO: 86 - protein, 5 aa, Homo sapiens
GGSGG

SEQ ID NO: 87 - protein, 5 aa, Homo sapiens
GSGSG

SEQ ID NO: 88 - protein, 5 aa, Homo sapiens
GSGGG

SEQ ID NO: 89 - protein, 5 aa, Homo sapiens
GGGSG

SEQ ID NO: 90 - protein, 5 aa, Homo sapiens
GSSSG

SEQ ID NO: 91 - protein, 5 aa, Homo sapiens
EAAAK

SEQ ID NO: 92 - protein, 10 aa, Homo sapiens
EAAAKEAAAK

SEQ ID NO: 93 - protein, 15 aa, Homo sapiens
EAAAKEAAAKEAAAK

SEQ ID NO: 94 - protein, 20 aa, Homo sapiens
EAAAKEAAAKEAAAKEAAAK

SEQ ID NO: 95 - protein, 25 aa, Homo sapiens
EAAAKEAAAKEAAAKEAAAKEAAAK

SEQ ID NO: 96 - protein, 30 aa, Homo sapiens
EAAAKEAAAKEAAAKEAAAKEAAAKEAAAK

SEQ ID NO: 97 - protein, 35 aa, Homo sapiens
EAAAKEAAAKEAAAKEAAAKEAAAKEAAAKEAAAK

SEQ ID NO: 98 - protein, 40 aa, Homo sapiens
EAAAKEAAAKEAAAKEAAAKEAAAKEAAAKEAAAKEAAAK

SEQ ID NO: 99 - protein, 45 aa, Homo sapiens
EAAAKEAAAKEAAAKEAAAKEAAAKEAAAKEAAAKEAAAKEAAAK

SEQ ID NO: 100 - protein, 50 aa, Homo sapiens
EAAAKEAAAKEAAAKEAAAKEAAAKEAAAKEAAAKEAAAKEAAAKEAAAK

SEQ ID NO: 101 - protein, 25 aa, Homo sapiens
LVGTDATLCCSFSPEPGFSLAQLNL

SEQ ID NO: 102 - protein, 25 aa, Homo sapiens
AQLNLIWQLTDTKQLVHSFAEGQDQ

SEQ ID NO: 103 - protein, 25 aa, Homo sapiens
EGQDQGSAYANRTALFPDLLAQGNA

SEQ ID NO: 104 - protein, 25 aa, Homo sapiens
AQGNASLRLQRVRVADEGSFTCFVS

SEQ ID NO: 105 - protein, 25 aa, Homo sapiens
TCFVSIRDFGSAAVSLQVAAPYSKP

SEQ ID NO: 106 - protein, 25 aa, Homo sapiens
PYSKPSMTLEPNKDLRPGDTVTITC

SEQ ID NO: 107 - protein, 25 aa, Homo sapiens
VTITCSSYRGYPEAEVFWQDGQGVP

SEQ ID NO: 108 - protein, 25 aa, Homo sapiens
GQGVPLTGNVTTSQMANEQGLFDVH

SEQ ID NO: 109 - protein, 25 aa, Homo sapiens
LFDVHSVLRVVLGANGTYSCLVRNP

SEQ ID NO: 110 - protein, 25 aa, Homo sapiens
LVRNPVLQQDAHGSVTITGQPMTFP

SEQ ID NO: 111 - DNA, 1389 bp, Homo sapiens
  source 1..1389: mol_type = other DNA
atgcaactcctgtcttgcattgcactaagtcttgcacttgtcacgaattcgcaggttcagctgcagcagtctggagctgaactggtaaagcctggggcttcagtgaagttgtcctgcaaggcttctggctacaccttcacaagttatgatataaactgggtgaggcagaggcctgaacagggacttgagtggattggatggatttttcctggagatgatactactaactacaatgagaatttcaagggcaaggccacactgactgcagacaaatcctccagcacagcctacatgcagctcaccaggctgacatctgaggactctgctgtctttttctgtgcaagatcggtgggctttgactactggggccaaggcaccactctcacagtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 112 - DNA, 690 bp, Homo sapiens
  source 1..690: mol_type = other DNA
atgcaactcctgtcttgcattgcactaagtcttgcacttgtcacgaattcacaaattgttctcatccagtctccagcaatcatgtctgcatctccaggggagaaggtcaccatgacctgcagtgccagttcaagtataagttacatgtactggtaccagcagaagccaggcacctcccccaaaagatggatttttgaaacatccaaactggcttctggagtccctgctcgcttcagtggcagtgggtctgggacctcttattctctcacaatcagcagcatggaggctgaagatgctgccacttattactgccatcagcggagtagttaccccacgttcggaggggggaccaagctggaaataaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 113 - protein, 462 aa, Homo sapiens
MQLLSCIALSLALVTNSQVQLQQSGAELVKPGASVKLSCKASGYTFTSYDINWVRQRPEQGLEWIGWIFPGDDTTNYNENFKGKATLTADKSSSTAYMQLTRLTSEDSAVFFCARSVGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 229 aa, Homo sapiens
MQLLSCIALSLALVTNSQIVLIQSPAIMSASPGEKVTMTCSASSSISYMYWYQQKPGTSPKRWIFETSKLASGVPARFSGSGSGTSYSLTISSMEAEDAATYYCHQRSSYPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 5 aa, Homo sapiens
GSGGS

SEQ ID NO: 116 - protein, 4 aa, Homo sapiens
GGGS

SEQ ID NO: 117 - protein, 534 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 118 - DNA, 21 bp, Homo sapiens
  source 1..21: mol_type = other DNA
aaaggccagtattaaggctag

(19) United States
(12) Patent Application Publication
(10) Pub. No.: US 2025/0144191 A1
(43) Pub. Date: May 8, 2025

(54) DENDRITIC CELL ASGPR TARGETING IMMUNOTHERAPEUTICS FOR MULTIPLE SCLEROSIS

(71) Applicant: BAYLOR RESEARCH INSTITUTE, Dallas, TX (US)

(72) Inventors: SangKon OH, Baltimore, MD (US); Gerard ZURAWSKI, Midlothian, TX (US)

(73) Assignee: BAYLOR RESEARCH INSTITUTE, Dallas, TX (US)

(21) Appl. No.: 19/010,936

(22) Filed: Jan. 6, 2025

Related U.S. Application Data
(63) Continuation of application No. 17/447,607, filed on Sep. 14, 2021, now Pat. No. 12,186,379.
Application No. 17/447,607 is a continuation of application No. 14/901,617, filed on Dec. 28, 2015, now abandoned.
(60) Provisional application No. 61/841,094, filed on Jun. 28, 2013.

(51) Int. Cl. A61K 39/00; A61P 25/00; A61P 25/28; A61P 37/02; C07K 14/46; C07K 16/28

(52) U.S. Cl. CPC A61K 39/0008; A61P 25/28; A61P 37/02; C07K 14/46; C07K 16/28; A61K 2039/505; A61K 2039/577; A61P 25/00; C07K 2319/33

(57) ABSTRACT

Methods and compositions for treating multiple sclerosis using dendritic cell anti-ASGPR antibodies fused to myelin basic protein or myelin oligodendrocyte glycoprotein are provided.

This application is a continuation of U.S. patent application Ser. No. 17/447,607, filed Sep. 14, 2021, which is a continuation of U.S. patent application Ser. No. 14/901,617, filed Dec. 28, 2015, which is a national phase application under 35 U.S.C. § 371 of International Application No. PCT/US2014/044711, filed Jun. 27, 2014, which claims the benefit of priority to U.S. Provisional Patent Application Ser. No. 61/841,094, filed Jun. 28, 2013, the entire contents of each of which are hereby incorporated by reference in their entirety.

The application contains a Sequence Listing prepared in compliance with ST.26 format and is hereby incorporated by reference in its entirety. Said Sequence Listing, created on Jan. 6, 2025 is named “BHCSP0380USC2.xml” and is 112,677 bytes in size.

### BACKGROUND OF THE INVENTION

### 1. Field of the Invention

The present invention relates generally to the field of medicine. More particularly, it concerns methods and compositions for treating multiple sclerosis using dendritic cell anti-ASGPR antibodies fused to myelin basic protein or myelin oligodendrocyte glycoprotein.

### 2. Description of Related Art

The inappropriate immune response of the body against substances and tissues normally present in the body is thought to give rise to autoimmune diseases (autoimmunity). Autoimmunity may be restricted to certain organs or involve a particular tissue in different places. While the treatment of autoimmune diseases is typically with immunosuppression-medication that decreases the immune response, the repertoire of these drugs can be limited and in some instances is insufficient to treat the underlying condition. As a large number of autoimmune diseases are recognized, treatment of these represents a substantial human health issue. Multiple sclerosis (MS), also known as disseminated sclerosis or encephalomyelitis disseminata, is an inflammatory disease in which myelin sheaths around axons of the brain and spinal cord are damaged, leading to loss of myelin and scarring. In some instances, the underlying mechanism is thought to be either destruction by the immune system. These changes affect the ability of nerve cells to communicate resulting in a wide range of signs and symptoms.

### SUMMARY OF THE INVENTION

Methods and compositions are provided that can be used to induce immune tolerance in autoimmune diseases or conditions. Specifically contemplated are immunotherapeutic compositions and methods of administering these compositions to patients. Embodiments are focused on compositions that target myelin sheath proteins or components to dendritic cells (DC) through receptor mediated endocytosis by targeting specific DC receptors with specific antibodies.

In some embodiments, a method of inducing immune tolerance to at least one myelin sheath protein in a patient is provided. In certain embodiments, the method comprises administering to the patient an effective amount of a composition comprising a dendritic cell targeting complex comprising a dendritic cell antibody, or targeting fragment thereof, attached to the at least one myelin sheath protein, or antigenic fragment thereof. In other embodiments, the myelin sheath protein is myelin basic protein (MBP), myelin oligodendrocyte glycoprotein (MOG), proteolipid protein (PLP), or myelin associated glycoprotein (MAG). In certain aspects the composition comprising a dendritic cell targeting complex comprising a dendritic cell antibody, or targeting fragment thereof, attached to the at least one myelin sheath protein, or antigenic fragment thereof is considered an immunotherapeutic.

In some embodiments, the immunotherapeutic comprises multiple myelin sheath proteins or myelin sheath components. In certain embodiments at least one myelin sheath protein of the immunotherapeutic is MBP. In other embodiments at least one myelin sheath protein of the immunotherapeutic is MOG.

In certain embodiments the dendritic cell antibody or fragment thereof of the dendritic cell targeting complex specifically binds asialoglycoprotein receptor (ASGPR). In yet other embodiments, dendritic cell targeting complex composition comprises multiple dendritic cell targeting complexes. In other aspects the multiple dendritic cell targeting complexes comprise different myelin sheath proteins or different antigenic fragments of one or more myelin sheath proteins. In still other aspects the myelin sheath protein or antigen fragment is separately attached to a dendritic cell antibody, or a targeting fragment thereof. In certain aspects dendritic cell antibody is attached to the myelin sheath protein using a peptide linker. There are different ways in which the antibody or antigenic fragment is attached to the myelin sheath protein. In some embodiments, they are attached directly. In certain embodiments, the antibody or antigenic fragment is attached to the myelin sheath protein through one or more covalent bonds. It is specifically contemplated that there may be a single polypeptide that includes the antibody or antigenic fragment and one or more myelin sheath proteins (or fragments thereof). Such a polypeptide may be described as a fusion protein if the two parts are attached covalently through peptide bonds (with or without a peptide linker). Such a fusion protein with an ASGPR antibody (or antibody fragment) and a myelin sheath protein (or fragment thereof) would not be naturally occurring.

Certain embodiments include an immunogenic composition comprising an isolated polypeptide comprising at least or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36. 37, 38, 39, 40, 41, 42,. 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 or more amino acids of SEQ ID NO: 42-53, including all values and ranges there between. In a further aspect the isolated polypeptide is a fusion protein. The composition can comprise an adjuvant. In certain aspects the isolated polypeptide is a fusion protein and/or a lipopeptide.

Embodiments include compositions that include a polypeptide, peptide, or protein that is or is at least 70%, 75%, 80%, 85%, 90%, 95%, 96%, 97%, 98%, or 99% identical or similar to any of SEQ ID NO: 42-53. Similar polypeptides, peptides and proteins, in some embodiments, are limited to those proteinaccous compounds whose substitutions are only with conservative amino acids. In other embodiments, only conservative substitutions are contemplated, while in others, deletions of nonessential amino acids or the addition of other amino acids in an area that is not involved in the compound's function are contemplated. In a further embodiment, a composition may include a polypeptide, peptide, or protein that is or is at least 70%, 75%, 80%, 85%, 90%, 95%, 96%, 97%, 98%, or 99% identical or similar to a ASGPR binding polypeptide, such as mAnti-ASGPR_49C11_7H (heavy chain) SEQ ID NO:42, mAnti-ASGPR_49C11_7K (light chain) SEQ ID NO:43, manti-hASGPR_6.3H9.1D11H (heavy chain) SEQ ID NO: 44, manti-hASGPR_6.3H9.1D11K (light chain) SEQ ID NO: 45, manti-hASGPR_5H8.1D4H (heavy chain) SEQ ID NO: 46, manti-hASGPR_5H8.1D4K (light chain) SEQ ID NO: 47, mAnti-ASGPR_4G2.2_ (heavy chain) SEQ ID NO: 48, mAnti-ASGPR_4G2.2_ (light chain) SEQ ID NO: 49, mAnti-ASGPR-5F10H (heavy chain) SEQ ID NO: 50, mAnti-ASGPR-5F10H (light chain) SEQ ID NO: 51, mAnti-ASGPR1H11 (heavy chain) SEQ ID NO: 52, or mAnti-ASGPR1H1 1 (light chain) SEQ ID NO: 53.

The ASGPR binding polypeptides described herein may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more variant amino acids within at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids, or any range derivable therein, of any of SEQ ID NO: 42-53.

In particular embodiments, the immunotherapeutic, DC targeting complex or ASGPR binding polypeptide is purified, which may be accomplished with or without minimal denaturation. In some aspects, the immunotherapeutic, DC targeting complex or ASGPR binding polypeptide is active, meaning the immunotherapeutic, DC targeting complex or ASGPR binding polypeptide retains some detectable level of function or activity, such as those described, including binding ability. It is contemplated that the immunotherapeutic, DC targeting complex or ASGPR binding polypeptide may be purified to about, at least about, or at most about 50, 55, 60, 65, 70, 75, 80, 85, 90, 95, 96, 97, 98, 99, 100% purity or homogeneity (with respect to other proteinaceous molecules and/or cellular macromolecules), or any range derivable therein. In additional embodiments, the recombinant immunotherapeutic, DC targeting complex or ASGPR binding polypeptide may be isolated. The term “isolated” can refer to a nucleic acid or polypeptide that is substantially free of cellular material, bacterial material, viral material, or culture medium (when produced by recombinant DNA techniques) of their source of origin, or chemical precursors or other chemicals (when chemically synthesized). Moreover, an isolated compound refers to one that can be administered to a subject as an isolated compound; in other words, the compound may not simply be considered “isolated” if it is adhered to a column or embedded in an agarose gel. Moreover, an “isolated nucleic acid fragment” or “isolated peptide” is a nucleic acid or protein fragment that is not naturally occurring as a fragment and/or is not typically in the functional state.

Furthermore, in certain embodiments of the current methods, methods may involve compositions containing about, at least about, or at most about 0.1, 0.2, 0.3, 0.4, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 21, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μg or mg of protein (or any range derivable therein). The protein may be in about, at least about, or at most about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 10, 11, 12, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μl or ml (or any range derivable therein). In certain aspects, one or more immunotherapeutics, DC targeting complexes or ASGPR binding polypeptides can be administered as a dose of 0.1, 0.2, 0.3, 0.4, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 21, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 mg per kg of body weight.

In particular embodiments, the immune tolerance response elicited by the immunotherapeutic or dendritic cell targeting complex may be complemented, supplemented, increased or augmented. In certain aspects the immune tolerance response elicited by the immunotherapeutic or dendritic cell targeting complex may be complemented, supplemented, increased or augmented by an adjuvant. In certain aspects the adjuvant is a tolerogenic adjuvant. In certain embodiments the immunotherapeutic or dendritic cell targeting complex composition further comprises at least one tolerogenic adjuvant. In certain aspects the tolerogenic adjuvant is attached to the dendritic cell targeting complex. In other aspects, the tolerogenic adjuvant is conjugated to the dendritic cell targeting complex. In still other aspects, the tolerogenic adjuvant is fused to the dendritic cell antibody, or targeting fragment thereof, and/or to the at least one myelin sheath protein. In specific embodiments, the tolerogenic adjuvant is selected from IL-10, dexamethasone, FK506 (Tacrolimus), cholera toxin B subunit, Escherichia coli heat-labile enterotoxin B subunit, IFN-beta, glucocorticoids, vitamin D3, and vitamin D3 analogues.

In particular embodiments, the use of binding polypeptides is contemplated to fuse, conjugate or bring together separate polypeptides, portions or modules of the immunotherapeutic or dendritic cell targeting complex. In certain aspects, the dendritic cell antibody or fragment thereof is attached to at least one myelin sheath protein through binding polypeptides. In specific embodiments, the binding polypeptides are dockerin and cohesin.

In certain aspects, administering to the patient an effective amount of a composition comprising a dendritic cell targeting complex comprises more than one administration of the composition. In certain aspects, the composition is administered orally, intravenously, subcutaneously, intradermally, intramuscularly, nasally, by injection, by inhalation, and/or using a nebulizer.

In particular aspects, the methods and compositions described are aimed at treating, preventing, ameliorating, suppressing, resolving, improving or otherwise addressing the symptoms of a subject or patient with an autoimmune disorder, disease or condition. In certain aspects, the subject exhibits one or more symptoms of a demyelinating disease. In other embodiments, the subject has been diagnosed with a demyelinating disease. In still other embodiments, the subject is at risk for a demyelinating disease. In specific embodiments, the demyelinating disease affects the central nervous system. In other specific embodiments, the demyelinating disease is an idiopathic inflammatory demyelinating disease. In certain aspects the demyelinating disease is multiple sclerosis, neuropathy, central pontine myelinolysis, tabes dorsalis, transverse myelitis, Devic's disease, progressive multifocal leukoencephalopathy, optic neuritis, or leukodystrophy. In some embodiments, the demyelinating disease is one of the borderline forms of multiple sclerosis. In some aspects, the borderline form of multiple sclerosis is standard multiple sclerosis, remitent-recidivant multiple sclerosis (RRMS), secondary progressive multiple sclerosis (SPMS), primary progressive multiple sclerosis (PPMS), KIR4.1 multiple sclerosis, optic-spinal multiple sclerosis, opticospinal multiple sclerosis, Devic's disease, acute disseminated encephalomyelitis (ADEM), acute hemorrhagic leukoencephalitis, Balo concentric sclerosis, Schilder disease, diffuse myelinoclastic sclerosis, Marburg multiple sclerosis, malignant multiple sclerosis, fulminant multiple sclerosis, acute multiple sclerosis, tumefactive multiple sclerosis, or solitary sclerosis. In yet other embodiments the demyelinating disease is Susac's syndrome, myalgic encephalomyelitis or leukoaraiosis.

In other specific embodiments, the demyelinating disease is multiple sclerosis. In certain aspects the demyelinating disease affects the peripheral nervous system. In additional embodiments the demyelinating disease is Guillain-Barré syndrome, chronic inflammatory demyelinating polyneuropathy, anti-MAG peripheral neuropathy, Charcot-Marie-Tooth Disease, copper deficiency, or progressive inflammatory neuropathy.

In certain aspects, the methods described comprising a dendritic cell targeting complex and/or immunotherapeutic further comprise preparing the composition. In other embodiments, the methods further comprise measuring antibodies against the at least one myelin sheath protein in the subject after administering the composition.

In some aspects, a method for treating a demyelinating disease in a subject comprising administering to the subject a pharmaceutically acceptable vaccine composition comprising at least a first ASGPR antibody, or binding fragment thereof, attached to myelin basic protein (MBP) and/or myelin oligodendrocyte glycoprotein (MOG), or antigenic fragment thereof is contemplated. In some embodiments, the ASGPR antibody, or binding fragment thereof, is fused to MBP or MOG, or an antigenic fragment thereof. In other embodiments, the subject is administered the vaccine composition multiple times. In still other embodiments, the composition is administered orally, intravenously, subcutaneously, intradermally, intramuscularly, nasally, by injection, by inhalation, and/or using a nebulizer. In certain aspects, the subject exhibits one or more symptoms of a demyelinating disease. In additional aspects, the subject has been diagnosed with a demyelinating disease. In some embodiments, the subject is at risk for a demyelinating disease. In other embodiments, the demyelinating disease affects the central nervous system. In additional embodiments, the demyelinating disease is an idiopathic inflammatory demyelinating disease. In certain aspects, the demyelinating disease is multiple sclerosis, neuropathy, central pontine myelinolysis, tabes dorsalis, transverse myelitis, Devic's disease, progressive multifocal leukoencephalopathy, optic neuritis, or leukodystrophy. In specific embodiments, the demyelinating disease is multiple sclerosis. In some embodiments, the demyelinating disease is one of the borderline forms of multiple sclerosis. In some aspects, the borderline form of multiple sclerosis is standard multiple sclerosis, remitent-recidivant multiple sclerosis (RRMS), secondary progressive multiple sclerosis (SPMS), primary progressive multiple sclerosis (PPMS), KIR4.1 multiple sclerosis, optic-spinal multiple sclerosis, opticospinal multiple sclerosis, Devic's disease, acute disseminated encephalomyelitis (ADEM), acute hemorrhagic leukoencephalitis, Balo concentric sclerosis, Schilder disease, diffuse myelinoclastic sclerosis, Marburg multiple sclerosis, malignant multiple sclerosis, fulminant multiple sclerosis, acute multiple sclerosis, tumefactive multiple sclerosis, or solitary sclerosis. In yet other embodiments the demyelinating disease is Susac's syndrome, myalgic encephalomyelitis or leukoaraiosis. In other aspects, the demyelinating disease affects the peripheral nervous system. In specific embodiments, the demyelinating disease is Guillain-Barré syndrome, chronic inflammatory demyelinating polyneuropathy, anti-MAG peripheral neuropathy, Charcot-Marie-Tooth Disease, copper deficiency, or progressive inflammatory neuropathy. In certain embodiments, the methods further comprise preparing the composition. In further embodiments still, the methods further comprise measuring antibodies against the at least one myelin sheath protein in the subject after administering the composition.

In some embodiments, a composition comprises at least a first ASGPR antibody, or binding fragment thereof, attached to myelin basic protein (MBP) and/or myelin oligodendrocyte glycoprotein (MOG), or antigenic fragment thereof. In other embodiments, the dendritic cell antibody is attached to the myelin sheath protein or antigenic fragment thereof using a peptide linker. In some embodiments, the composition further comprises at least one tolerogenic adjuvant. In still other embodiments, the tolerogenic adjuvant is attached to the dendritic cell targeting complex. In additional embodiments, the tolerogenic adjuvant is conjugated to the dendritic cell targeting complex. In some aspects, the tolerogenic adjuvant is fused to the dendritic cell antibody, or targeting fragment thereof, and/or to the at least one myelin sheath protein. In specific aspects, the tolerogenic adjuvant is selected from IL-10, dexamethasone, FK506 (Tacrolimus), cholera toxin B subunit, Escherichia coli heat-labile enterotoxin B subunit, IFN-beta, glucocorticoids, vitamin D3, and vitamin D3 analogues. In still other aspects, the dendritic cell antibody is attached to at least one myelin sheath protein or antigenic fragment thereof through binding polypeptides. In some embodiments, the binding polypeptides are dockerin and cohesin.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the embodiments will become apparent from the following detailed description. It should be understood, however, that the detailed description and the specific examples, while indicating preferred embodiments, are given by way of illustration only, since various changes and modifications within the spirit and scope of the embodiments will become apparent to those skilled in the art from this detailed description.

### BRIEF DESCRIPTION OF THE DRAWINGS

The following drawings form part of the present specification and are included to further demonstrate certain aspects of the embodiments. Certain embodiments may be better understood by reference to one or more of these drawings in combination with the detailed description of specific embodiments presented herein.

FIG. 1A-B: (A) Experimental method schematic. (B) Anti-DC-ASGPR-PSA vaccine can prime PSA-specific IL-10-producing CD4+ T cells in NHPs.

FIG. 2A-B: (A) Experimental method schematic. (B) Anti-DC-ASGPR-HA1 promotes IL-10-producing HA1-specific CD4+ T cells in vivo.

FIG. 3A-B: Monoclonal antibody selection. (A) RT-PCR assay. (B) Luminex assay. 3/7 clones induce DCs to express IL-10, other clones induce less than 10 pg/ml IL-10. Levels of IL-10 expression are variable among donors, IL-6 and TNFa are in background levels in Luminex assay.

FIG. 4: Monoclonal antibody selection. 49C11 binds to CD11c⁺ blood DCs in MS patients and healthy donors.

FIG. 5: Generation of anti-DC-ASGPR-MBP and anti-DC-ASGPR-MOG fusion proteins. Expression product of anti-DC-ASGPR (49C11) mAb fused to MBP and MOG.

FIG. 6A-B: (A) Experimental method schematic. (B) In vitro validation of anti-DC-ASGPR (49C11)-MBP proteins. Anti-DC-ASGPR (49C11)-MBP binds to CD11c⁺ DCs in PBMCs and promotes MBP-specific Treg responses.

FIG. 7A-B: In vitro validation of anti-DC-ASGPR (49C11)-MOG proteins. (A) CD11c+ DC staining. (B) MS patient PBMC loading assay. Anti-DC-ASGPR-MOG binds to DCs and promotes MOG-specific Treg response.

FIG. 8: Effectiveness of anti-ASGPR-hMOG in EAE Model in Cynomolgus Macaque-Experimental Design.

FIG. 9: Anti-DC-ASGPR-MOG treatment suppresses EAE development progression

FIG. 10: Anti-DC-ASGPR-MOG treatment results in enhanced survival of animals

FIG. 11: Magnetic resonance images of AM637 animal brain on day 22.

### DESCRIPTION OF ILLUSTRATIVE EMBODIMENTS

### I. MULTIPLE SCLEROSIS IMMUNOTHERAPEUTIC TECHNOLOGY

Methods and compositions are provided to generate immune tolerance in a subject against one or more myelin sheath antigens in order to protect against or treat for autoimmune disorders such as multiple sclerosis. Dendritic cells (DCs) are antigen-presenting cells that play a key role in regulating antigen-specific immunity (Mellman and Steinman 2001), (Banchereau, Briere et al. 2000), (Cella, Sallusto et al. 1997). DCs capture antigens, process them into peptides, and present these to T cells. Therefore delivering antigens directly to DC is a focus area for developing immunotherapeutics.

Provided herein are immunotherapeutics compositions containing myelin sheath antigens for delivery to DC in order to generate an immune tolerance response to myelin sheath protein or components or to suppress an immune response to myelin sheath protein or components. In some embodiments the myelin sheath protein or component is myelin basic protein (MBP). In other embodiments the myelin sheath protein or component is myelin oligodendrocyte glycoprotein (MOG). In yet other embodiments, the myelin sheath protein or component is proteolipid protein (PLP). In still other embodiments the myelin sheath protein or component is myelin associated glycoprotein. In additional embodiments, the myelin sheath protein or component is any one of peripheral myelin protein (PMP-22), Po protein, connexin 32 protein, Schwann cell myelin protein, or oligodendrocyte-myelin glycoprotein (OMgp). In still additional embodiments, the immunotherapeutic comprises multiple different myelin sheath components as stated above.

The type of immune response that is generated after delivery of an immunotherapeutic may be modulated by the type of receptor that is targeted by said therapeutic. In some embodiments of the present methods, asialoglycoprotein receptor (ASGPR) is targeted by ASGPR binding antibodies. In some aspects the antibodies are monoclonal antibodies. In yet other aspects, the antibodies are mouse monoclonal antibodies.

In still other aspects, the antibodies are human/mouse chimeras. In further aspects, the antibodies are humanized monoclonal antibodies.

In certain aspects, the type of receptor that is targeted by the immunotherapeutic is DEC-205. DEC-205 is a type I cell surface protein expressed primarily by dendritic cells (DC). In some embodiments of the present methods, DEC-205 is targeted by DEC-205 binding antibodies. In some aspects the antibodies are DEC-205 monoclonal antibodies. In yet other aspects, the antibodies are DEC-205 mouse monoclonal antibodies. In still other aspects, the antibodies are DEC-205 mouse/human chimeras. In further aspects, the antibodies are humanized DEC-205 mouse monoclonal antibodies.

Such technology and embodiments are described in the following U.S. Patent Publications 20120282281 (Agents that Engage Antigen-Presenting Cells Through Dendritic Cell Asialoglycoprotein Receptor (DC-ASGPR)); 20120244155 (Dendritic Cells (DCs) Targeting for Tuberculosis (TB) Vaccine); 20120237513 (Vaccines Based on Targeting Antigen to DCIR Expressed on Antigen-Presenting Cells); 20120231023 (Novel Vaccine Adjuvants Based on Targeting Adjuvants to Antibodies Directly to Antigen-Presenting Cells); 20120213768 (Diagnostic and Therapeutic Uses for B Cell Maturation Antigen); 20120128710 (Enhancement of Pathogen-Specific Memory Th17 Cell Responses); 20120121592 (Targeting Antigens to Human Dendritic Cells Via DC-Asialoglycoprotein Receptor to Produce IL-10 Regulatory T-Cells; 20120039916 (NOVEL VACCINE ADJUVANTS BASED ON TARGETING ADJUVANTS TO ANTIBODIES DIRECTLY TO ANTIGEN-PRESENTING CELLS); 20120035240 (CONSERVED HBV AND HCV SEQUENCES USEFUL FOR GENE SILENCING); 20120020990 (ISOLATED MAMMALIAN MONOCYTE CELL GENES; RELATED REAGENTS); 20120004643 (Vaccines Based on Targeting Antigen to DCIR Expressed on Antigen-Presenting Cells); 20110274653 (DENDRITIC CELL IMMUNORECEPTORS (DCIR)-MEDIATED CROSSPRIMING OF HUMAN CD8+ T CELLS); 20110081343 (VACCINES DIRECTED TO LANGERHANS CELLS); 20100330115 (Multivariable Antigens Complexed with Targeting Humanized Monoclonal Antibody); 20100322929 (ANTIGEN PRESENTING CELL TARGETED CANCER VACCINES); 20100297114 (ANTIGEN PRESENTING CELL TARGETED VACCINES); 20100291082 (ANTIGEN PRESENTING CELL TARGETED ANTI-VIRAL VACCINES); 20100239575 (ANTI-CD40 ANTIBODIES AND USES THEREOF); 20100209907 (ISOLATED MAMMALIAN MONOCYTE CELL

GENES; RELATED REAGENTS); 20100135994 (HIV VACCINE BASED ON TARGETING MAXIMIZED GAG AND NEF TO DENDRITIC CELLS); 20080267984(Activation of Human Antigen-Presenting Cells Through Dendritic Cell Lectin-Like Oxidized LDL Receptor-1 (LOX-1)); 20080254047 (Activation of Human Antigen-Presenting Cells Through CLEC-6); 20080254044 (Multivariable Antigens Complexed with Targeting Humanized Monoclonal Antibody); 20080241170 (Vaccines Based on Targeting Antigen to DCIR Expressed on Antigen-Presenting Cells); 20080233140 (Therapeutic Applications of Activation of Human Antigen-Presenting Cells Through Dectin-1); 20080206262 (Agents That Engage Antigen-Presenting Cells Through Dendritic Cell Asialoglycoprotein Receptor (DC-ASGPR)); 20080070854 (Conserved Hbv and Hcv Sequences Useful for Gene Silencing); 20050287582 (Antibodies that specifically bind to FDF03); 20050059808 (Isolated mammalian monocyte cell genes; related reagents); 20040143858 (Isolated mammalian monocyte cell genes; related reagents); 20030105303 (Isolated mammalian monocyte cell genes; related reagents); and, 20020161218 (Hepatitis C virus vaccine), all of which are hereby incorporated by reference.

### II. NUCLEIC ACIDS

In certain embodiments, there are recombinant nucleic acids encoding the proteins, polypeptides, or peptides described herein. Polynucleotides contemplated for use in methods and compositions include those encoding antibodies against DC receptors (also referred to as anti-DC antibodies and DC targeting antibodies) or binding portions thereof.

As used in this application, the term “polynucleotide” refers to a nucleic acid molecule that either is recombinant or has been isolated free of total genomic nucleic acid. Included within the term “polynucleotide” are oligonucleotides (nucleic acids 100 residues or fewer in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein (see above).

In particular embodiments, there are isolated nucleic acid segments and recombinant vectors incorporating nucleic acid sequences that encode a polypeptide (e.g., an antibody or fragment thereof) that binds to DC receptors. The term “recombinant” may be used in conjunction with a polypeptide or the name of a specific polypeptide, and this generally refers to a polypeptide produced from a nucleic acid molecule that has been manipulated in vitro or that is a replication product of such a molecule.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the case of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

In certain embodiments, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

### Vectors

Polypeptides may be encoded by a nucleic acid molecule. The nucleic acid molecule can be in the form of a nucleic acid vector. The term “vector” is used to refer to a carrier nucleic acid molecule into which a heterologous nucleic acid sequence can be inserted for introduction into a cell where it can be replicated and expressed. A nucleic acid sequence can be “heterologous,” which means that it is in a context foreign to the cell in which the vector is being introduced or to the nucleic acid in which is incorporated, which includes a sequence homologous to a sequence in the cell or nucleic acid but in a position within the host cell or nucleic acid where it is ordinarily not found. Vectors include DNAs, RNAs, plasmids, cosmids, viruses (bacteriophage, animal viruses, and plant viruses), and artificial chromosomes (e.g., YACs). One of skill in the art would be well equipped to construct a vector through standard recombinant techniques (for example Sambrook et al., 2001; Ausubel et al., 1996, both incorporated herein by reference). Vectors may be used in a host cell to produce an antibody that binds a dendritic cell receptor.

The term “expression vector” refers to a vector containing a nucleic acid sequence coding for at least part of a gene product capable of being transcribed. In some cases, RNA molecules are then translated into a protein, polypeptide, or peptide. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described herein.

### Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

### Expression Systems

Numerous expression systems exist that comprise at least a part or all of the compositions discussed above. Prokaryote-and/or eukaryote-based systems can be employed for use with an embodiment to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. No. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

In addition to the disclosed expression systems, other examples of expression systems include STRATAGENER's COMPLETE CONTROL Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

### III. PROTEINACEOUS COMPOSITIONS

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Proteins may be recombinant, or synthesized in vitro. Alternatively, a non-recombinant or recombinant protein may be isolated from bacteria. It is also contemplated that bacteria containing such a variant may be implemented in compositions and methods. Consequently, a protein need not be isolated.

The term “functionally equivalent codon” is used herein to refer to codons that encode the same amino acid, such as the six codons for arginine or serine, and also refers to codons that encode biologically equivalent amino acids (see Table, below).

Codon Table

| Amino Acids | | | Codons |
|---|---|---|---|
| Alanine | Ala | A | GCA GCC GCG GCU |
| Cysteine | Cys | C | UGC UGU |
| Aspartic | Asp | D | GAC GAU |
| acid | | | |
| Glutamic | Glu | E | GAA GAG |
| acid | | | |
| Phenyl- | Phe | F | UUC UUU |
| alanine | | | |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

It also will be understood that amino acid and nucleic acid sequences may include additional residues, such as additional N- or C-terminal amino acids, or 5′ or 3′ sequences, respectively, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. It is understood that an amino acid can be substituted for another having a similar hydrophilicity value and still produce a biologically equivalent and immunologically equivalent protein.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known and include: arginine and lysine; glutamate and aspartate; serine and threonine; glutamine and asparagine; and valine, leucine and isoleucine.

It is contemplated that in compositions there is between about 0.001 mg and about 10 mg of total polypeptide, peptide, and/or protein per ml. Thus, the concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein). Of this, about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% may be an antibody that targets DC, and may be used in combination with other proteins, antibodies or protein-binding antibodies described herein.

### Polypeptides and Polypeptide Production

Embodiments involve polypeptides, peptides, and proteins and immunogenic fragments thereof for use in various aspects described herein. For example, specific antibodies are assayed for or used in binding to DC receptors and presenting myelin sheath protein or components as antigens. In specific embodiments, all or part of proteins described herein can also be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Alternatively, recombinant DNA technology may be employed wherein a nucleotide sequence that encodes a peptide or polypeptide is inserted into an expression vector, transformed or transfected into an appropriate host cell and cultivated under conditions suitable for expression.

One embodiment includes the use of gene transfer to cells, including microorganisms, for the production and/or presentation of proteins. The gene for the protein of interest may be transferred into appropriate host cells followed by culture of cells under the appropriate conditions. A nucleic acid encoding virtually any polypeptide may be employed. The generation of recombinant expression vectors, and the elements included therein, are discussed herein. Alternatively, the protein to be produced may be an endogenous protein normally synthesized by the cell used for protein production.

In a certain aspects a DC receptor fragment comprises substantially all of the extracellular domain of a protein which has at least 85% identity, at least 90% identity, at least 95% identity, or at least 97-99% identity, including all values and ranges there between, to a sequence selected over the length of the fragment sequence.

Also included in immunogenic compositions are fusion proteins composed of myelin sheath protein or components, or immunogenic fragments of myelin sheath protein or components (e.g., myelin basic protein, proteolipid protein, myelin-associated glycoprotein, myelin oligodendrocyte glycoprotein, peripheral myelin protein (PMP-22), Po protein, connexin 32 protein, Schwann cell myelin protein, oligodendrocyte-myelin glycoprotein (OMgp)). Alternatively, embodiments also include individual fusion proteins of myelin sheath protein or components or immunogenic fragments thereof, as a fusion protein with heterologous sequences such as a provider of T-cell epitopes or purification tags, for example: β-galactosidase, glutathione-S-transferase, 6×His, green fluorescent proteins (GFP), epitope tags such as FLAG, myc tag, poly histidine, or viral surface proteins such as influenza virus haemagglutinin, or bacterial proteins such as tetanus toxoid, diphtheria toxoid, CRM197.

### Antibodies and Antibody-Like Molecules

In certain aspects, one or more antibodies or antibody-like molecules (e.g., polypeptides comprising antibody CDR domains) may be obtained or produced which have a specificity for a DC receptor. These antibodies may be used in various diagnostic or therapeutic applications described herein.

As used herein, the term “antibody” is intended to refer broadly to any immunologic binding agent such as IgG, IgM, IgA, IgD and IgE as well as polypeptides comprising antibody CDR domains that retain antigen binding activity. Thus, the term “antibody” is used to refer to any antibody-like molecule that has an antigen binding region, and includes antibody fragments such as Fab′, Fab, F(ab′)2, single domain antibodies (DABs), Fv, scFv (single chain Fv), and polypeptides with antibody CDRs, scaffolding domains that display the CDRs (e.g., anticalins) or a nanobody. For example, the nanobody can be antigen-specific VHH (e.g., a recombinant VHH) from a camelid IgG2 or IgG3, or a CDR-displaying frame from such camelid Ig. Production and use of camelid antibodies is described in EP1118669 A9 and EP1414858 B1, both of which are incorporated herein by reference. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

“Mini-antibodies” or “minibodies” are also contemplated for use with embodiments. Minibodies are sFv polypeptide chains which include oligomerization domains at their C-termini, separated from the sFv by a hinge region (Pack et al., 1992). The oligomerization domain comprises self-associating α-helices, e.g., leucine zippers, that can be further stabilized by additional disulfide bonds. The oligomerization domain is designed to be compatible with vectorial folding across a membrane, a process thought to facilitate in vivo folding of the polypeptide into a functional binding protein. Generally, minibodies are produced using recombinant methods well known in the art. See, e.g., Pack et al. (1992); Cumber et al. (1992).

Antibody-like binding peptidomimetics are also contemplated in embodiments. Liu et al. (2003) describe “antibody like binding peptidomimetics” (ABiPs), which are peptides that act as pared-down antibodies and have certain advantages of longer serum half-life as well as less cumbersome synthesis methods.

Alternative scaffolds for antigen binding peptides, such as CDRs are also available and can be used to generate DC receptor-binding molecules in accordance with the embodiments. Generally, a person skilled in the art knows how to determine the type of protein scaffold on which to graft at least one of the CDRs arising from the original antibody.

More particularly, it is known that to be selected such scaffolds must meet the greatest number of criteria as follows (Skerra, 2000): good phylogenetic conservation; known three-dimensional structure (as, for example, by crystallography, NMR spectroscopy or any other technique known to a person skilled in the art); small size; few or no post-transcriptional modifications; and/or easy to produce, express and purify.

The origin of such protein scaffolds can be, but is not limited to, the structures selected among: fibronectin and preferentially fibronectin type III domain 10, lipocalin, anticalin (Skerra, 2001), thioredoxin A or proteins with a repeated motif such as the “ankyrin repeat” (Kohl et al., 2003), the “armadillo repeat”, the “leucine-rich repeat” and the “tetratricopeptide repeat”. For example, anticalins or lipocalin derivatives are a type of binding proteins that have affinities and specificities for various target molecules; such proteins are described in US Patent Publication Nos. 20100285564, 20060058510, 20060088908, 20050106660, and PCT Publication No. WO2006/056464, incorporated herein by reference.

Scaffolds derived from toxins such as, for example, toxins from scorpions, insects, plants, mollusks, etc., and the protein inhibiters of neuronal NO synthase (PIN) may also be used in certain aspects.

Monoclonal antibodies (MAbs) are recognized to have certain advantages, e.g., reproducibility and large-scale production. Embodiments include monoclonal antibodies of the human, murine, monkey, rat, hamster, rabbit and chicken origin.

“Humanized” antibodies are also contemplated, as are chimeric antibodies

from mouse, rat, or other species, bearing human constant and/or variable region domains, bispecific antibodies, recombinant and engineered antibodies and fragments thereof. As used herein, the term “humanized” immunoglobulin refers to an immunoglobulin comprising a human framework region and one or more CDR's from a non-human (usually a mouse or rat) immunoglobulin. The non-human immunoglobulin providing the CDR's is called the “donor” and the human immunoglobulin providing the framework is called the “acceptor”. A “humanized antibody” is an antibody comprising a humanized light chain and a humanized heavy chain immunoglobulin. In order to describe antibodies of some embodiments, the strength with which an antibody molecule binds an epitope, known as affinity, can be measured. The affinity of an antibody may be determined by measuring an association constant (Ka) or dissociation constant (Kd). Antibodies deemed useful in certain embodiments may have an association constant of about, at least about, or at most about 10c6, 10e7, 10e8,10e9 or 10c10 M or any range derivable therein. Similarly, in some embodiments antibodies may have a dissociation constant of about, at least about or at most about 10e-6, 10e-7, 10e-8, 10e-9 or 10e-10. M or any range derivable therein. These values are reported for antibodies discussed herein and the same assay may be used to evaluate the binding properties of such antibodies.

In certain embodiments, the antibodies are recombinant antibodies. A recombinant antibody differs from an endogenously-produced antibody. For example, recombinant antibodies differ with respect to their glycosylation status (see, for example, Jefferis, R. “Glycosylation of Recombinant Antibody Therapeutics” Biotechnol. Prog. 2005, 21:11-16 which is herein incorporated by reference).

In certain embodiments, a polypeptide that specifically binds to DC receptors is able to bind a DC receptor on the surface of the cells and present a myelin sheath protein or component that allows the generation of a robust immune tolerance to that myelin sheath protein or component. Moreover, in some embodiments, the polypeptide that is used can provide immune tolerance against a myelin sheath protein or component and protect against multiple sclerosis.

### 1. Methods for Generating Antibodies

Methods for generating antibodies (e.g., monoclonal antibodies and/or monoclonal antibodies) are known in the art. Briefly, a polyclonal antibody is prepared by immunizing an animal with a DC receptor polypeptide or a portion thereof in accordance with embodiments and collecting antisera from that immunized animal.

A wide range of animal species can be used for the production of antisera. Typically the animal used for production of antisera is a rabbit, a mouse, a rat, a hamster, a guinea pig or a goat. The choice of animal may be decided upon the case of manipulation, costs or the desired amount of sera, as would be known to one of skill in the art. It will be appreciated that antibodies can also be produced transgenically through the generation of a mammal or plant that is transgenic for the immunoglobulin heavy and light chain sequences of interest and production of the antibody in a recoverable form therefrom. In connection with the transgenic production in mammals, antibodies can be produced in, and recovered from, the milk of goats, cows, or other mammals. See, e.g., U.S. Pat. Nos. 5,827,690, 5,756,687, 5,750,172, and 5,741,957.

As is also well known in the art, the immunogenicity of a particular immunogen composition can be enhanced by the use of non-specific stimulators of the immune response, known as adjuvants. Suitable adjuvants include any acceptable immunostimulatory compound, such as cytokines, chemokines, cofactors, toxins, plasmodia, synthetic compositions or vectors encoding such adjuvants. Adjuvants may be chemically conjugated to antibodies or antigen-delivering antibody fusions proteins. Alternatively adjuvants may be recombinantly fused to antibodies or antigen-delivering antibody fusions proteins. In certain aspects, adjuvants may be chemically conjugated or recombinantly fused to cohesin or dockerin to allow for binding to any other molecule containing a corresponding dockerin or cohesin binding domain.

Adjuvants that may be used in accordance with embodiments include, but are not limited to, IL-1, IL-2, IL-4, IL-7, IL-12, -interferon, GMCSP, BCG, aluminum hydroxide, Poly ICLC, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion is also contemplated. MHC antigens may even be used. Exemplary adjuvants may include complete Freund's adjuvant (a non-specific stimulator of the immune response containing killed Mycobacterium tuberculosis), incomplete Freund's adjuvants and/or aluminum hydroxide adjuvant.

In addition to adjuvants, it may be desirable to coadminister biologic response modifiers (BRM), which have been shown to upregulate T cell immunity or downregulate suppressor cell activity. Such BRMs include, but are not limited to, Cimetidine (CIM; 1200 mg/d) (Smith/Kline, PA); low-dose Cyclophosphamide (CYP; 300 mg/m2) (Johnson/Mead, NJ), cytokines such as -interferon, IL-2, or IL-12 or genes encoding proteins involved in immune helper functions, such as B-7.

The amount of immunogen composition used in the production of antibodies varies upon the nature of the immunogen as well as the animal used for immunization. A variety of routes can be used to administer the immunogen including but not limited to subcutaneous, intramuscular, intradermal, intraepidermal, intravenous and intraperitoneal. The production of antibodies may be monitored by sampling blood of the immunized animal at various points following immunization.

A second, booster dose (e.g., provided in an injection), may also be given. The process of boosting and titering is repeated until a suitable titer is achieved. When a desired level of immunogenicity is obtained, the immunized animal can be bled and the serum isolated and stored, and/or the animal can be used to generate MAbs.

For production of rabbit polyclonal antibodies, the animal can be bled through an car vein or alternatively by cardiac puncture. The removed blood is allowed to coagulate and then centrifuged to separate serum components from whole cells and blood clots. The serum may be used as is for various applications or else the desired antibody fraction may be purified by well-known methods, such as affinity chromatography using another antibody, a peptide bound to a solid matrix, or by using, e.g., protein A or protein G chromatography, among others.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified protein, polypeptide, peptide or domain, be it a wild-type or mutant composition. The immunizing composition is administered in a manner effective to stimulate antibody producing cells.

The methods for generating monoclonal antibodies (MAbs) generally begin along the same lines as those for preparing polyclonal antibodies. In some embodiments, Rodents such as mice and rats are used in generating monoclonal antibodies. In some embodiments, rabbit, sheep or frog cells are used in generating monoclonal antibodies. The use of rats is well known and may provide certain advantages (Goding, 1986, pp. 60 61). Mice (e.g., BALB/c mice) are routinely used and generally give a high percentage of stable fusions.

The animals are injected with antigen, generally as described above. The antigen may be mixed with adjuvant, such as Freund's complete or incomplete adjuvant. Booster administrations with the same antigen or DNA encoding the antigen may occur at approximately two-week intervals. As discussed in the Examples, the antigen may be altered compared to an antigen sequence found in nature.

Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Generally, spleen cells are a rich source of antibody-producing cells that are in the dividing plasmablast stage. Typically, peripheral blood cells may be readily obtained, as peripheral blood is easily accessible.

In some embodiments, a panel of animals will have been immunized and the spleen of an animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody producing B lymphocytes from the immunized animal are then fused with cells of an immortal myeloma cell, generally one of the same species as the animal that was immunized. Myeloma cell lines suited for use in hybridoma producing fusion procedures preferably are non-antibody producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65 66, 1986; Campbell, pp. 75 83, 1984). cites). For example, where the immunized animal is a mouse, one may use P3 X63/Ag8, X63 Ag8.653, NS1/1.Ag 4 1, Sp210 Ag14, FO, NSO/U, MPC 11, MPC11 X45 GTG 1.7 and $194/5XX0 Bul; for rats, one may use R210.RCY3, Y3 Ag 1.2.3, IR983F and 4B210; and U 266, GM1500 GRG2, LICR LON HMy2 and UC729 6 are all useful in connection with human cell fusions. See Yoo et al. (2002), for a discussion of myeloma expression systems.

One murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8 azaguanine resistant mouse murine myeloma SP2/0 non producer cell line.

Methods for generating hybrids of antibody producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al., (1977). The use of electrically induced fusion methods is also appropriate (Goding pp. 71 74, 1986).

Fusion procedures usually produce viable hybrids at low frequencies, about 1×10-6 to 1×10-8. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

A selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody producing cell lines, which clones can then be propagated indefinitely to provide MAbs. The cell lines may be exploited for MAb production in two basic ways. First, a sample of the hybridoma can be injected (often into the peritoneal cavity) into a histocompatible animal of the type that was used to provide the somatic and myeloma cells for the original fusion (e.g., a syngeneic mouse). Optionally, the animals are primed with a hydrocarbon, especially oils such as pristane (tetramethylpentadecane) prior to injection. The injected animal develops tumors secreting the specific monoclonal antibody produced by the fused cell hybrid. The body fluids of the animal, such as serum or ascites fluid, can then be tapped to provide MAbs in high concentration. Second, the individual cell lines could be cultured in vitro, where the MAbs are naturally secreted into the culture medium from which they can be readily obtained in high concentrations.

Further, expression of antibodies (or other moieties therefrom) from production cell lines can be enhanced using a number of known techniques. For example, the glutamine synthetase and DHFR gene expression systems are common approaches for enhancing expression under certain conditions. High expressing cell clones can be identified using conventional techniques, such as limited dilution cloning and microdrop technology. The GS system is discussed in whole or part in connection with European Patent Nos. 0 216 846, 0 256 055, and 0 323 997 and European Patent Application No. 89303964.4.

MAbs produced by either means may be further purified, if desired, using filtration, centrifugation and various chromatographic methods such as HPLC or affinity chromatography. Fragments of the monoclonal antibodies can be obtained from the monoclonal antibodies so produced by methods which include digestion with enzymes, such as pepsin or papain, and/or by cleavage of disulfide bonds by chemical reduction. Alternatively, monoclonal antibody fragments can be synthesized using an automated peptide synthesizer.

It is also contemplated that a molecular cloning approach may be used to generate monoclonal antibodies. In one embodiment, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 10e4 times as many antibodies can be produced and screened in a single round, and that new specificities are generated by H and L chain combination which further increases the chance of finding appropriate antibodies.

Another embodiment concerns producing antibodies, for example, as is found in U.S. Pat. No. 6,091,001, which describes methods to produce a cell expressing an antibody from a genomic sequence of the cell comprising a modified immunoglobulin locus using Cre-mediated site-specific recombination is disclosed. The method involves first transfecting an antibody-producing cell with a homology-targeting vector comprising a lox site and a targeting sequence homologous to a first DNA sequence adjacent to the region of the immunoglobulin loci of the genomic sequence which is to be converted to a modified region, so the first lox site is inserted into the genomic sequence via site-specific homologous recombination. Then the cell is transfected with a lox-targeting vector comprising a second lox site suitable for Cre-mediated recombination with the integrated lox site and a modifying sequence to convert the region of the immunoglobulin loci to the modified region. This conversion is performed by interacting the lox sites with Cre in vivo, so that the modifying sequence inserts into the genomic sequence via Cre-mediated site-specific recombination of the lox sites.

Alternatively, monoclonal antibody fragments can be synthesized using an automated peptide synthesizer, or by expression of full-length gene or of gene fragments in E. coli.

It is further contemplated that monoclonal antibodies may be further screened or optimized for properties relating to specificity, avidity, half-life, immunogenicity, binding association, binding disassociation, or overall functional properties relative to being a treatment for infection and/or disease state. Thus, it is contemplated that monoclonal antibodies may have 1, 2, 3, 4, 5, 6, or more alterations in the amino acid sequence of 1, 2, 3, 4, 5, or 6 CDRs of monoclonal antibodies mAnti-ASGPR 49C11, mAnti-ASGPR 4G2.2, mAnti-ASGPR 5F10, mAnti-ASGPR 1H11, mAnti-ASGPR 6.3H9.1D11, mAnti-ASGPR 5H8.1D4. It is contemplated that the amino acid in position 1, 2, 3, 4, 5, 6, 7, 8, 9, or 10 of CDR1, CDR2, CDR3, CDR4, CDR5, or CDR6 of the VJ or VDJ region of the light or heavy variable region of monoclonal antibodies mAnti-ASGPR 49C11, mAnti-ASGPR 4G2.2, mAnti-ASGPR 5F10, mAnti-ASGPR 1H11, mAnti-ASGPR 6.3H9.1D11, mAnti-ASGPR 5H8.1D4, may have an insertion, deletion, or substitution with a conserved or non-conserved amino acid. Such amino acids that can either be substituted or constitute the substitution are disclosed above.

Methods of determining CDRs from the sequence of a variable region are known in the art (see, for example, Zhao and Lu, “A germline knowledge based computational approach for determining antibody complementarity determining regions.” Mol. Immunol., (2010) 47(4):694-700, which is herein incorporated by reference).

In some embodiments, fragments of a whole antibody can perform the function of binding antigens. Examples of binding fragments are (i) the Fab fragment constituted with the VL, VH, CL and CHI domains; (ii) the Fd fragment consisting of the VH and CHI domains; (iii) the Fv fragment constituted with the VL and VH domains of a single antibody; (iv) the dAb fragment (Ward, 1989; McCafferty et al., 1990; Holt et al., 2003), which is constituted with a VH or a VL domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., 1988; Huston et al., 1988); (viii) bispecific single chain Fv dimers (PCT/US92/09965) and (ix) “diabodies”, multivalent or multispecific fragments constructed by gene fusion (WO94/13804; Holliger et al., 1993). Fv, scFv or diabody molecules may be stabilized by the incorporation of disulphide bridges linking the VH and VL domains (Reiter et al., 1996). Minibodies comprising a scFv joined to a CH3 domain may also be made (Hu et al. 1996). The citations in this paragraph are all incorporated by reference.

Antibodies also include bispecific antibodies. Bispecific or bifunctional antibodies form a second generation of monoclonal antibodies in which two different variable regions are combined in the same molecule (Holliger, P. & Winter, G. 1999 Cancer and metastasis rev. 18:411-419, 1999). Their use has been demonstrated both in the diagnostic field and in the therapy field from their capacity to recruit new effector functions or to target several molecules on the surface of tumor cells. Where bispecific antibodies are to be used, these may be conventional bispecific antibodies, which can be manufactured in a variety of ways (Holliger et al, PNAS USA 90:6444-6448, 1993), e.g. prepared chemically or from hybrid hybridomas, or may be any of the bispecific antibody fragments mentioned above. These antibodies can be obtained by chemical methods (Glennie et al., 1987 J. Immunol. 139, 2367-2375; Repp et al., J. Hemat. 377-382, 1995) or somatic methods (Staerz

U. D. and Bevan M. J. PNAS 83, 1986; et al., Method Enzymol. 121:210-228, 1986) but likewise by genetic engineering techniques which allow the heterodimerization to be forced and thus facilitate the process of purification of the antibody sought (Merchand et al. Nature Biotech, 16:677-681, 1998). Examples of bispecific antibodies include those of the BiTE™ technology in which the binding domains of two antibodies with different specificity can be used and directly linked via short flexible peptides. This combines two antibodies on a short single polypeptide chain. Diabodies and scFv can be constructed without an Fc region, using only variable domains, potentially reducing the effects of anti-idiotypic reaction. The citations in this paragraph are all incorporated by reference.

Bispecific antibodies can be constructed as entire IgG, as bispecific Fab′2, as Fab′PEG, as diabodies or else as bispecific scFv. Further, two bispecific antibodies can be linked using routine methods known in the art to form tetravalent antibodies.

Bispecific diabodies, as opposed to bispecific whole antibodies, may also be particularly useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against a DC receptor, then a library can be made where the other arm is varied and an antibody of appropriate specificity selected. Bispecific whole antibodies may be made by alternative engineering methods as described in Ridgeway et al, (Protein Eng., 9:616-621, 1996), which is hereby incorporated by reference.

### Antibody and Polypeptide Conjugates

Embodiments provide antibodies and antibody-like molecules against DC receptors, polypeptides and peptides that are linked to at least one agent to form an antibody conjugate or payload or fusion. Embodiments also provide antibody drug conjugates (ADC). In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, therapeutic enzymes, antibiotics, radio-labeled nucleotides and the like. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to which they are attached to be detected, and/or further quantified if desired.

Antibody conjugates are in certain embodiments used as diagnostic agents. Antibody diagnostics generally fall within two classes, those for use in in vitro diagnostics, such as in a variety of immunoassays, and/or those for use in vivo diagnostic protocols, generally known as “antibody directed imaging”. Many appropriate imaging agents are known in the art, as are methods for their attachment to antibodies (see, for e.g., U.S. Pat. Nos. 5,021,236; 4,938,948; and 4,472,509, each incorporated herein by reference). The imaging moieties used can be paramagnetic ions; radioactive isotopes; fluorochromes; NMR-detectable substances; X-ray imaging.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might use astatine²¹¹, carbon¹⁴, chromium⁵¹, chlorine³⁶, cobalt⁵⁷, cobalt⁵⁸, copper⁶⁷, Eu¹⁵², gallium⁶⁷, hydrogen³, iodine¹²³, iodine¹²⁵, iodine¹³¹, indium¹¹¹, iron⁵⁹, phosphorus³², rhenium¹⁸⁶, rhenium¹⁸⁸, selenium⁷⁵, sulphur³⁵, technicium⁹⁹) and/or yttrium⁹⁰. ¹²⁵I is often used in certain embodiments, and technicium⁹⁹ and/or indium¹¹¹ are also often used due to their low energy and suitability for long range detection. Radioactively labeled monoclonal antibodies may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies may be labeled with technetium99m by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red, among others.

Antibody conjugates include those intended primarily for use in vitro, where the antibody is linked to a secondary binding ligand and/or to an enzyme (an enzyme tag) that will generate a colored product upon contact with a chromogenic substrate. Examples of suitable enzymes include, but are not limited to, urease, alkaline phosphatase, (horseradish) hydrogen peroxidase or glucose oxidase. Preferred secondary binding ligands are biotin and/or avidin and streptavidin compounds. The use of such labels is well known to those of skill in the art and are described, for example, in U.S. Pat. No. 3,817,837; 3,850,752; 3,939,350; 3,996,345; 4,277,437; 4,275,149 and 4,366,241; each incorporated herein by reference.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter & Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; and Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In some embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

### IV. DENDRITIC CELL IMMUNOTHERAPEUTICS

As used herein, “Dendritic Cells” (DCs) refers to any member of a diverse population of morphologically similar cell types found in lymphoid or non-lymphoid tissues. These cells are characterized by their distinctive morphology, high levels of surface MHC-class II expression (Steinman, et al., Ann. Rev. Immunol. 9:271 (1991); incorporated herein by reference for its description of such cells). These cells can be isolated from a number of tissue sources, and conveniently, from peripheral blood, as described herein.

### Myelin Sheath Proteins and Components as Antigens

In certain embodiments any myelin sheath protein or component (including but not limited to myelin sheath protein, glycoprotein, lipid or glycolipid) may be recombinantly fused or chemically conjugated to a DC targeting antibody to deliver the myelin sheath protein or component to a dendritic cell. A myelin sheath protein or component may be any myelin sheath protein or component that when fused to a DC targeting antibody is sufficient to evoke an immune tolerance response in a subject. In certain embodiments the immune response is sufficient to protect a subject from multiple sclerosis. In other embodiments protection afforded by the antigen/targeting antibody fusion is sufficient to depress or prevent symptoms associated with multiple sclerosis.

In some embodiments the myelin sheath protein or component is myelin basic protein (MBP). In other embodiments the myelin sheath protein or component is myelin oligodendrocyte glycoprotein (MOG). In yet other embodiments, the myelin sheath protein or component is proteolipid protein (PLP). In still other embodiments the myelin sheath protein or component is myelin associated glycoprotein. In additional embodiments, the myelin sheath protein or component is any one of peripheral myelin protein (PMP-22), Po protein, connexin 32 protein, Schwann cell myelin protein, or oligodendrocyte-myelin glycoprotein (OMgp). In still additional embodiments, the immunotherapeutic comprises multiple different myelin sheath components as stated above.

### Dendritic Cell Specific Antibodies

In certain aspects, antibodies used to target myelin sheath protein or components to dendritic cells are dendritic cell specific antibodies. Some of the antibodies that may be used for this purpose are known in the art.

In some embodiments anti-ASGPR antibodies are used to target myelin sheath protein or components to dendritic cells. One example includes anti-dendritic cell immunoreceptor monoclonal antibody conjugates, wherein the conjugate comprises antigenic peptides that are loaded or chemically coupled to the antibody. Such antibodies are described in U.S. Pat. No. 8,236,934, incorporated herein by reference.

### Peptide Linkers

In certain aspects, peptide linkers are used to link dendritic cell specific antibodies and myelin sheath protein or components to be presented. Peptide linkers may incorporate glycosylation sites or introduce secondary structure. Additionally these linkers increase the efficiency of expression or stability of the fusion protein and as a result the efficiency of antigen presentation to a dendritic cell. Linkers may include SSVSPTTSVHPTPTSVPPTPTKSSP (SEQ ID NO:1); PTSTPADSSTITPTATPTATPTIKG (SEQ ID NO:2); TVTPTATATPSAIVTTITPTATTKP (SEQ ID NO:3); or TNGSITVAATAPTVTPTVNATPSAA (SEQ ID NO:4). These examples and others are discussed in WO 2010/104747, the contents of which are incorporated herein by reference. Additional linkers useful for this purpose are described in US 2010/291082, the contents of which are incorporated herein by reference.

In certain aspects antibody domains, adjuvants antigens or peptide linkers may be bound by high-affinity interacting protein domains. In some embodiments a high-affinity interacting protein domains involves a cohesin-dockerin binding pair. A cohesin-dockerin binding pair may be recombinantly fused to an antibody domain, adjuvants, antigens or peptide linkers. In some aspects the dockerin is modified such that it is capable of binding to a cohesin domain when recombinantly encoded in an internal (non carboxy or non-amino terminal end) portion of a polypeptide. In certain aspects the linker region is not a peptide linker. An example of a non-peptide linker region may result as the product of chemical conjugation wherein the covalent bond that is formed between molecules is not a peptide bond.

### Adjuvants

In other embodiments an immune adjuvant is directly fused or otherwise linked to the dendritic cell specific antibody in order to enhance the efficacy of the immunotherapeutic. In certain aspects the immune adjuvant may be a toll-like receptor (TLR) agonist. TLR agonists comprise flagellins from Salmonella enterica or Vibrio cholerae. In certain aspects the adjuvant in Flagellin-1 or Flagellin-2. TLR agonists may be specific for certain TLR classes (i.e., TLR5, TLR7 or TLR9 agonists) and may be presented in any combination or as any modification. Examples of such immune adjuvants are described in WO 2012/021834, the contents of which are incorporated herein by reference. Poly ICLC, a TLR3 ligand is also contemplated for use with myelin sheath protein or component DC targeting immunotherapeutic compositions. In some embodiments the DC targeting immunotherapeutic comprises myelin basic protein (MBP), myelin oligodendrocyte glycoprotein (MOG), proteolipid protein (PLP), myelin associated glycoprotein, peripheral myelin protein (PMP-22), Po protein, connexin 32 protein, Schwann cell myelin protein, or oligodendrocyte-myelin glycoprotein (OMgp) and Poly ICLC is delivered separately from the antibody antigen fusion polypeptide. In still additional embodiments, the immunotherapeutic comprises one or more different myelin sheath components as stated above. In one embodiment, the Poly ICLC is as described in U.S. Pat. No. 7,439,349, the contents of which are incorporated herein by reference. In one embodiment, the Poly ICLC is Hiltonol®. Interleukins are also contemplated as adjuvants that may be fused to a dendritic cell specific antibody or to a protein domain capable of binding with high affinity to a corresponding or complementary domain on a dendritic cell specific antibody. Non-limiting examples of such interleukins are IL-21, IL-2, IL-9 and IL-10. In some embodiments the interleukin proteins are human interleukins. In certain aspects the adjuvant is an HLA-DR antigen-associated invariant chain that augments antigen processing. In certain aspects the adjuvant is interferon alpha. In yet other embodiments the adjuvant is a toxin that will deliver a death signal to cells also receiving an myelin sheath protein or component, thereby augmenting immunotherapeutic efficiency. One example of such a toxin is PE38. Any adjuvant may be delivered in fused or conjugated form with a DC targeting immunotherapeutic or may be delivered concomitantly as part of the same composition or preparation without fusion or direct conjugation.

### Tolerogenic Adjuvants

In certain embodiments the immune adjuvant may be a tolerogenic adjuvant. In certain instances a tolerogenic adjuvant may refer to an adjuvant that is utilized for tolerogenic immunization, where the aim of immunization with an antigen is to generate an immune response such that the antigen is tolerated by an immunized subject. In certain aspects, the goal of a tolerogenic adjuvant is to enhance tolerogenic immunization such that tolerance to an antigen is further enhanced. In certain embodiments a tolerogenic adjuvant is used to tolerize autoimmunity. In yet other aspects, a tolerogenic adjuvant is used to tolerize harmful autoimmunity. In some embodiments the tolerogenic adjuvant is an immunosuppressant. In yet other embodiments the tolerogenic adjuvant is dexamethasone, FK506 (Tacrolimus), cholera toxin B subunit, Escherichia coli heat-labile enterotoxin B subunit, IFN-beta, glucocorticoids, vitamin D3, or vitamin D3 analogues. In certain aspects the tolerogenic adjuvant is administered concurrently with h a DC targeting immunotherapeutic. In other aspects a tolerogenic adjuvant is administered before or after administration of a DC targeting immunotherapeutic. In yet other embodiments two or more tolerogenic adjuvants are administered concurrently, before or after administration of a DC targeting immunotherapeutic. In certain aspects, the tolerogenic adjuvant may be fused, conjugated or otherwise linked to the DC targeting immunotherapeutic. In one embodiment, the tolerogenic adjuvant is interleukin-10 (IL-10). In another embodiment IL-10 is co-administered with the DC targeting immunotherapeutic. In certain aspects, IL-10 is fused by recombinant methods. In other aspects IL-10 is conjugated. In other embodiments IL-10 is linked by coupling or other modular domains.

### Constructs

The sequences given below, when presented as antibody H or L chain or protein secreted by mammalian cells are shown as amino acids without signal peptide (i.e., as ‘mature’ secreted protein), while the DNA sequences are the entire coding region including signal sequences if present.

All examples of H chain constructs are typically used in co-transfection of CHO cells with matching L chain vectors. Also, in some embodiments immunotherapeutics will have humanized variable regions, which have been described for anti-ASGPR_49C11, anti-CD40 12E12, anti-Langerin 15B10, anti-DCIR 9E8, and anti-LOX-1 15C4.

Anti-ASGPR heavy chain and light chains may be selected from the following:

### Anti-DC ASGPR mAbs

[mAnti-ASGPR-49C11-7H-LV-hIgG4H-C]

(SEQ ID NO.: 5)
DVQLQESGPDLVKPSQSLSLTCTVTGYSITSGYSWHWIRQFPGNKLEWMG
YILESGSTNYNPSLKSRISITRDTSKNQFFLQLNSVTTEDTATYFCARSN
YGSFASWGQGTLVTVSAAKTTGPSVFPLAPCSRSTSESTAALGCLVKDYF
PEPVTVSWNSGALTSGVHTFPAVLQSSGLYSLSSVVTVPSSSLGTKTYTC
NVDHKPSNTKVDKRVESKYGPPCPPCPAPEFEGGPSVFLFPPKPKDTLMI
SRTPEVTCVVVDVSQEDPEVQFNWYVDGVEVHNAKTKPREEQFNSTYRVV
SVLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDGS
FFLYSRLTVDKSRWQEGNVFSCSVMHEALHNHYTQKSLSLSLGKAS.

The above sequence is a chimera between the H chain variable region of the mAb 49C11 (shown underlined) and the C region of hIgG4.

[mAnti-ASGPR-49C11-7K-LV-hIgGK-C] is the corresponding L chain chimera (sequence below, variable region underlined)

(SEQ ID NO.: 6)
QIVLTQSPAIMSASPGEKVTMTCSASSSVSHMHWYQQKSGTSPKRWIYDT
SRLASGVPARFSGSGSGTSYSLTISSMEAEDAATYYCQQWSSHPWSFGGG
TKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC.

[mAnti-ASGPR-4G2.2_Hv-V-hIgG4H-C]

(SEQ ID NO.: 7)
QIQLVQSGPELKKPGETVKISCKASGYTFTNYGMNWVKQVPGKGLRWMGW
MDTFTGEPTYADDFKGRFAFSLETSASTAYLQINSLKNEDTATYFCARGG
ILRLNYFDYWGQGTTLTVSSAKTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEFEGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGKAS.

The above sequence is a chimera between the H chain variable of the mAb 4G2.2 (shown underlined) and the C region of hIgG4.

[mAnti-ASGPR-4G2.2_Kv-V-hIgGK-C] is the corresponding L chain chimera (sequence below, variable region underlined)

(SEQ ID NO.: 8)
DIQMTQSSSSFSVSLGDRVTITCKASEDIYNRLGWYQQKPGNAPRLLISG
ATSLETGVPSRFSGSGSGKDYALSITSLQTEDLATYYCQQCWTSPYTFGG
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

[mAnti-ASGPR-5F10H-LV-hIgG4H-C] is

(SEQ ID NO.: 9)
EVQLQQSGPELVKPGASVKMSCKASGYTFTDYYMKWVKQSHGKSLEWIGD
INPNYGDTFYNQKFEGKATLTVDKSSRTAYMQLNSLTSEDSAVYYCGRGD
YGYFDVWGAGTTVTVSSAKTKGPSVFPLAPCSRSTSESTAALGCLVKDYF
PEPVTVSWNSGALTSGVHTFPAVLQSSGLYSLSSVVTVPSSSLGTKTYTC
NVDHKPSNTKVDKRVESKYGPPCPPCPAPEFEGGPSVFLFPPKPKDTLMI
SRTPEVTCVVVDVSQEDPEVQFNWYVDGVEVHNAKTKPREEQFNSTYRVV
SVLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDGS
FFLYSRLTVDKSRWQEGNVFSCSVMHEALHNHYTQKSLLSLGKAS.

The above sequence is a chimera between the H chain variable of the mAb 5F10H (shown underlined) and the C region of hIgG4.

[mAnti-ASGPR-5F10K-LV-hIgGK-C] is the corresponding L chain chimera (sequence below, variable region underlined)

(SEQ ID NO.: 10)
DIVMTQSHKFMSTSVGDRVSITCKASQDVGTAVAWYQQKPGQSPKLLIYW
ASTRHTGVPDRFTGSGSGTDFTLTINNVQSEDLADYFCQQYSSNPYMFGG
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

[mAnti-ASGPR1H11H-V-hIgG4H-C] is

(SEQ ID NO.: 11)
QLQQSGPELVKPGASVKISCKTSGYTFTEYTMHWVRQSHGKSLEWIGGIN
PINGGPTYNQKFKGKATLTVDKSSSTAYMELRSLTSEDSAVYYCARWDYG
SRDVMDYWGQGTSVTVSSAKTKGPSVFPLAPCSRSTSESTAALGCLVKDY
FPEPVTVSWNSGALTSGVHTFPAVLQSSGLYSLSSVVTVPSSSLGTKTYT
CNVDHKPSNTKVDKRVESKYGPPCPPCPAPEFEGGPSVFLFPPKPKDTLM
ISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLGKAS.

The above sequence is a chimera between the H chain variable of the mAb 1H11 (shown underlined) and the C region of hIgG4.

[mAnti-ASGPR1H11K-LV-hIgGK-C] is the corresponding L chain chimera (sequence below, variable region underlined)

(SEQ ID NO.: 12)
NIVMTQSPKSMSMSVGERVTLSCKASENVGTYVSWYQQRPEQSPKLLIYG
ASNRYTGVPDRFTGSGSATDFTLTISSVQAEDLADYHCGQTYSYIFTFGS
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

Examples of full length DC targeting antibody/antigen constructs are as follows:

mAnti-ASGPR_49C11_7H-LV-hIgG4H-C-Flex-v1-hMBP (LV-hIgG4H-C sequence underlined, Flex-v1 sequence in bold, hMBP sequence double underlined)

(SEQ ID NO: 13)
ATGAGAGCGCTGATTCTTTTGTGCCTGTTCACAGCCTTTCCTGGTATCCTGTCTGA
TGTGCAGCTTCAGGAGTCAGGACCTGACCTGGTGAAACCTTCTCAGTCACTTTCA
CTCACCTGCACTGTCACTGGCTACTCCATCACCAGTGGTTATAGCTGGCACTGGA
TCCGGCAGTTTCCAGGAAACAAACTGGAATGGATGGGCTACATACTCTTCAGTGG
TAGCACTAACTACAACCCATCTCTGAAAAGTCGAATCTCTATCACTCGAGACACA
TCCAAGAACCAGTTCTTCCTGCAGTTGAATTCTGTGACTACTGAGGACACAGCCA
CATATTTCTGTGCAAGATCTAACTATGGTTCCTTTGCTTCCTGGGGCCAAGGGACT
CTGGTCACTGTCTCTGCAGCCAAAACAACGGGCCCATCCGTCTTCCCCCTGGCGC
CCTGCTCCAGGAGCACCTCCGAGAGCACAGCCGCCCTGGGCTGCCTGGTCAAGG
ACTACTTCCCCGAACCGGTGACGGTGTCGTGGAACTCAGGCGCCCTGACCAGCG
GCGTGCACACCTTCCCGGCTGTCCTACAGTCCTCAGGACTCTACTCCCTCAGCAG
CGTGGTGACCGTGCCCTCCAGCAGCTTGGGCACGAAGACCTACACCTGCAACGT
AGATCACAAGCCCAGCAACACCAAGGTGGACAAGAGAGTTGAGTCCAAATATGG
TCCCCCATGCCCACCCTGCCCAGCACCTGAGTTCGAAGGGGGACCATCAGTCTTC
CTGTTCCCCCCAAAACCCAAGGACACTCTCATGATCTCCCGGACCCCTGAGGTCA
CGTGCGTGGTGGTGGACGTGAGCCAGGAAGACCCCGAGGTCCAGTTCAACTGGT
ACGTGGATGGCGTGGAGGTGCATAATGCCAAGACAAAGCCGCGGGAGGAGCAG
TTCAACAGCACGTACCGTGTGGTCAGCGTCCTCACCGTCCTGCACCAGGACTGGC
TGAACGGCAAGGAGTACAAGTGCAAGGTCTCCAACAAAGGCCTCCCGTCCTCCA
TCGAGAAAACCATCTCCAAAGCCAAAGGGCAGCCCCGAGAGCCACAGGTGTACA
CCCTGCCCCCATCCCAGGAGGAGATGACCAAGAACCAGGTCAGCCTGACCTGCC
TGGTCAAAGGCTTCTACCCCAGCGACATCGCCGTGGAGTGGGAGAGCAATGGGC
AGCCGGAGAACAACTACAAGACCACGCCTCCCGTGCTGGACTCCGACGGCTCCT
TCTTCCTCTACAGCAGGCTAACCGTGGACAAGAGCAGGTGGCAGGAGGGGAATG
TCTTCTCATGCTCCGTGATGCATGAGGCTCTGCACAACCACTACACACAGAAGAG
CCTCTCCCTGTCTCTGGGTAAAGCTAGTCAGACCCCCACCAACACCATCAGCGTG
ACCCCCACCAACAACAGCACCCCCACCAACAACAGCAACCCCAAGCCCAACCCC
GCTAGTGCATCACAAAAGCGGCCTTCACAACGGCACGGATCTAAATATCTGGCG
ACAGCCTCTACCATGGATCACGCCAGGCATGGCTTTCTGCCCAGGCACAGAGAT
ACTGGAATCTTGGACTCCATCGGCAGGTTCTTTGGCGGCGACCGAGGGGCTCCCA
AGAGAGGGAGTGGCAAGGATAGCCATCATCCAGCCCGAACAGCCCACTACGGA
AGCCTGCCGCAGAAAAGCCACGGTCGCACGCAGGATGAAAATCCCGTTGTGCAC
TTCTTCAAAAACATTGTGACCCCACGAACTCCTCCACCTTCCCAAGGCAAGGGCA
GAGGTCTCAGTCTCAGCCGGTTCAGTTGGGGGGCCGAGGGCCAGAGACCCGGAT
TTGGTTATGGGGGAAGGGCTAGCGACTACAAGTCTGCACATAAGGGGTTCAAAG
GGGTCGACGCACAGGGAACCCTGTCCAAAATATTTAAGCTTGGTGGCCGCGACT
CCCGCTCAGGCTCTCCCATGGCTCGGCGCTGA
(SEQ ID NO: 14)
DVQLQESGPDLVKPSQSLSLTCTVTGYSITSGYSWHWIRQFPGNKLEWMGYILFSGS
TNYNPSLKSRISITRDTSKNQFFLQLNSVTTEDTATYFCARSNYGSFASWGQGTLVTV
SAAKTTGPSVFPLAPCSRSTSESTAALGCLVKDYFPEPVTVSWNSGALTSGVHTFPAV
LQSSGLYSLSSVVTVPSSSLGTKTYTCNVDHKPSNTKVDKRVESKYGPPCPPCPAPEF
EGGPSVFLFPPKPKDTLMISRTPEVTCVVVDVSQEDPEVQFNWYVDGVEVHNAKTK
PREEQFNSTYRVVSVLTVLHQDWLNGKEYKCKVSNKGLPSSIEKTISKAKGQPREPQ
VYTLPPSQEEMTKNQVSLTCLVKGFYPSDIAVEWESNGQPENNYKTTPPVLDSDGSF
FLYSRLTVDKSRWQEGNVESCSVMHEALHNHYTQKSLSLSLGKASQTPTNTISVTP
TNNSTPTNNSNPKPNPASASQKRPSQRHGSKYLATASTMDHARHGELPRHRDTGIL
DSIGRFFGGDRGAPKRGSGKDSHHPARTAHYGSLPQKSHGRTQDENPVVHFFKNIVT
PRTPPPSQGKGRGLSLSRESWGAEGQRPGFGYGGRASDYKSAHKGFKGVDAQGTLS
KIFKLGGRDSRSGSPMARR
mAnti-ASGPR_49C11_7K-LV-hIgGK-C
(LV-hIgGK-C sequence underlined)
(SEQ ID NO: 15)
ATGGATTTTCAAGTGCAGATTTTCAGCTTCCTGCTAATCAGTGCCTCAGTCATAAT
ATCCAGAGGACAAATTGTTCTCACCCAGTCTCCAGCAATCATGTCTGCATCTCCA
GGGGAGAAGGTCACCATGACCTGCAGTGCCAGCTCAAGTGTAAGTCACATGCAC
TGGTACCAGCAGAAGTCAGGCACTTCCCCCAAAAGATGGATTTATGACACATCC
AGACTGGCTTCTGGAGTCCCTGCTCGCTTCAGTGGCAGTGGGTCTGGGACCTCTT
ACTCTCTCACAATCAGCAGCATGGAGGCTGAAGATGCTGCCACTTATTACTGCCA
GCAGTGGAGTAGTCACCCATGGTCGTTCGGTGGAGGCACCAAACTCGAGATCAA
ACGAACTGTGGCTGCACCATCTGTCTTCATCTTCCCGCCATCTGATGAGCAGTTG
AAATCTGGAACTGCCTCTGTTGTGTGCCTGCTGAATAACTTCTATCCCAGAGAGG
CCAAAGTACAGTGGAAGGTGGATAACGCCCTCCAATCGGGTAACTCCCAGGAGA
GTGTCACAGAGCAGGACAGCAAGGACAGCACCTACAGCCTCAGCAGCACCCTGA
CGCTGAGCAAAGCAGACTACGAGAAACACAAAGTCTATGCCTGCGAAGTCACCC
ATCAGGGCCTGAGCTCGCCCGTCACAAAGAGCTTCAACAGGGGAGAGTGTTAG
(SEQ ID NO: 16)
QIVLTQSPAIMSASPGEKVTMTCSASSSVSHMHWYQQKSGTSPKRWIYDTSRLASGV
PARFSGSGSGTSYSLTISSMEAEDAATYYCQQWSSHPWSFGGGTKLEIKRTVAAPSV
FIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC
mAnti-ASGPR_49C11_7H-LV-hlgG4H-C-hMOG
(LV-hlgG4H-C sequence underlined; hMOG sequence bold)
(SEQ ID NO: 17)
ATGAGAGCGCTGATTCTTTTGTGCCTGTTCACAGCCTTTCCTGGTATCCTGTCTGA
TGTGCAGCTTCAGGAGTCAGGACCTGACCTGGTGAAACCTTCTCAGTCACTTTCA
CTCACCTGCACTGTCACTGGCTACTCCATCACCAGTGGTTATAGCTGGCACTGGA
TCCGGCAGTTTCCAGGAAACAAACTGGAATGGATGGGCTACATACTCTTCAGTGG
TAGCACTAACTACAACCCATCTCTGAAAAGTCGAATCTCTATCACTCGAGACACA
TCCAAGAACCAGTTCTTCCTGCAGTTGAATTCTGTGACTACTGAGGACACAGCCA
CATATTTCTGTGCAAGATCTAACTATGGTTCCTTTGCTTCCTGGGGCCAAGGGACT
CTGGTCACTGTCTCTGCAGCCAAAACAACGGGCCCATCCGTCTTCCCCCTGGCGC
CCTGCTCCAGGAGCACCTCCGAGAGCACAGCCGCCCTGGGCTGCCTGGTCAAGG
ACTACTTCCCCGAACCGGTGACGGTGTCGTGGAACTCAGGCGCCCTGACCAGCG
GCGTGCACACCTTCCCGGCTGTCCTACAGTCCTCAGGACTCTACTCCCTCAGCAG
CGTGGTGACCGTGCCCTCCAGCAGCTTGGGCACGAAGACCTACACCTGCAACGT
AGATCACAAGCCCAGCAACACCAAGGTGGACAAGAGAGTTGAGTCCAAATATGG
TCCCCCATGCCCACCCTGCCCAGCACCTGAGTTCGAAGGGGGACCATCAGTCTTC
CTGTTCCCCCCAAAACCCAAGGACACTCTCATGATCTCCCGGACCCCTGAGGTCA
CGTGCGTGGTGGTGGACGTGAGCCAGGAAGACCCCGAGGTCCAGTTCAACTGGT
ACGTGGATGGCGTGGAGGTGCATAATGCCAAGACAAAGCCGCGGGAGGAGCAG
TTCAACAGCACGTACCGTGTGGTCAGCGTCCTCACCGTCCTGCACCAGGACTGGC
TGAACGGCAAGGAGTACAAGTGCAAGGTCTCCAACAAAGGCCTCCCGTCCTCCA
TCGAGAAAACCATCTCCAAAGCCAAAGGGCAGCCCCGAGAGCCACAGGTGTACA
CCCTGCCCCCATCCCAGGAGGAGATGACCAAGAACCAGGTCAGCCTGACCTGCC
TGGTCAAAGGCTTCTACCCCAGCGACATCGCCGTGGAGTGGGAGAGCAATGGGC
AGCCGGAGAACAACTACAAGACCACGCCTCCCGTGCTGGACTCCGACGGCTCCT
TCTTCCTCTACAGCAGGCTAACCGTGGACAAGAGCAGGTGGCAGGAGGGGAATG
TCTTCTCATGCTCCGTGATGCATGAGGCTCTGCACAACCACTACACACAGAAGAG
CCTCTCCCTGTCTCTGGGTAAAGCTAGTGGTCAGTTTAGAGTCATTGGGCCCAGA
CACCCTATAAGGGCTCTTGTGGGAGACGAGGTCGAGCTGCCGTGTCGCATTAGTC
CAGGCAAAAACGCCACAGGGATGGAAGTGGGGTGGTACAGGCCTCCCTTCTCTA
GGGTTGTGCATCTCTACCGCAACGGCAAAGATCAGGATGGAGATCAAGCTCCTG
AATATCGGGGCCGGACTGAGCTGCTCAAGGACGCGATCGGCGAGGGTAAGGTGA
CCTTGCGCATCCGAAATGTTAGATTCAGCGATGAAGGCGGATTTACGTGCTTCTT
TCGGGACCACTCATACCAGGAGGAAGCCGCAATGGAACTGAAGGTGGAGGACCC
CTTCTATTGGGTATCCCCAGCTAGCTGA
(SEQ ID NO: 18)
DVQLQESGPDLVKPSQSLSLTCTVTGYSITSGYSWHWIRQFPGNKLEWMGYILFSGS
TNYNPSLKSRISITRDTSKNQFFLQLNSVTTEDTATYFCARSNYGSFASWGQGTLVTV
SAAKTTGPSVFPLAPCSRSTSESTAALGCLVKDYFPEPVTVSWNSGALTSGVHTFPAV
LQSSGLYSLSSVVTVPSSSLGTKTYTCNVDHKPSNTKVDKRVESKYGPPCPPCPAPEF
EGGPSVFLFPPKPKDTLMISRTPEVTCVVVDVSQEDPEVQFNWYVDGVEVHNAKTK
PREEQFNSTYRVVSVLTVLHQDWLNGKEYKCKVSNKGLPSSIEKTISKAKGQPREPQ
VYTLPPSQEEMTKNQVSLTCLVKGFYPSDIAVEWESNGQPENNYKTTPPVLDSDGSF
FLYSRLTVDKSRWQEGNVFSCSVMHEALHNHYTQKSLSLSLGKASGQFRVIGPRHP
IRALVGDEVELPCRISPGKNATGMEVGWYRPPFSRVVHLYRNGKDQDGDQAPE
YRGRTELLKDAIGEGKVTLRIRNVRFSDEGGFTCFFRDHSYQEEAAMELKVEDP
FYWVSPAS
mAnti-ASGPR_49C11_7K-LV-hIgGK-C
(LV-hIgGK-C sequence underlined)
(SEQ ID NO: 19)
ATGGATTTTCAAGTGCAGATTTTCAGCTTCCTGCTAATCAGTGCCTCAGTCATAAT
ATCCAGAGGACAAATTGTTCTCACCCAGTCTCCAGCAATCATGTCTGCATCTCCA
GGGGAGAAGGTCACCATGACCTGCAGTGCCAGCTCAAGTGTAAGTCACATGCAC
TGGTACCAGCAGAAGTCAGGCACTTCCCCCAAAAGATGGATTTATGACACATCC
AGACTGGCTTCTGGAGTCCCTGCTCGCTTCAGTGGCAGTGGGTCTGGGACCTCTT
ACTCTCTCACAATCAGCAGCATGGAGGCTGAAGATGCTGCCACTTATTACTGCCA
GCAGTGGAGTAGTCACCCATGGTCGTTCGGTGGAGGCACCAAACTCGAGATCAA
ACGAACTGTGGCTGCACCATCTGTCTTCATCTTCCCGCCATCTGATGAGCAGTTG
AAATCTGGAACTGCCTCTGTTGTGTGCCTGCTGAATAACTTCTATCCCAGAGAGG
CCAAAGTACAGTGGAAGGTGGATAACGCCCTCCAATCGGGTAACTCCCAGGAGA
GTGTCACAGAGCAGGACAGCAAGGACAGCACCTACAGCCTCAGCAGCACCCTGA
CGCTGAGCAAAGCAGACTACGAGAAACACAAAGTCTATGCCTGCGAAGTCACCC
ATCAGGGCCTGAGCTCGCCCGTCACAAAGAGCTTCAACAGGGGAGAGTGTTAG
(SEQ ID NO: 20)
QIVLTQSPAIMSASPGEKVTMTCSASSSVSHMHWYQQKSGTSPKRWIYDTSRLASGV
PARFSGSGSGTSYSLTISSMEAEDAATYYCQQWSSHPWSFGGGTKLEIKRTVAAPSV
FIFPPSDEQLKSGTASVVCLLNNFYPREAKVQWKVDNALQSGNSQESVTEQDSKDST
YSLSSTLTLSKADYEKHKVYACEVTHQGLSSPVTKSENRGEC
6xHis-Cohesin-hMOG
(6xHis underlined; Cohesin in bold; hMOG double underlined)
(SEQ ID NO: 21)
ATGGATCCCAAAGGATCCCTTTCCTGGAGAATACTTCTGTTTCTCTCCCTGGCTTT
TGAGTTGAGCTACGGACTCGACATCACATCCCACCATCACCATCACCATGACGAT
CTGGATGCAGTAAGGATTAAAGTGGACACAGTAAATGCAAAACCGGGAGACACA
GTAAGAATACCTGTAAGATTCAGCGGTATACCATCCAAGGGAATAGCAAACTGT
GACTTTGTATACAGCTATGACCCGAATGTACTTGAGATAATAGAGATAGAACCG
GGAGACATAATAGTTGACCCGAATCCTGACAAGAGCTTTGATACTGCAGTATATC
CTGACAGAAAGATAATAGTATTCCTGTTTGCAGAAGACAGCGGAACAGGAGCGT
ATGCAATAACTAAAGACGGAGTATTTGCTACGATAGTAGCGAAAGTAAAAGAAG
GAGCACCTAACGGACTCAGTGTAATCAAATTTGTAGAAGTAGGCGGATTTGCGA
ACAATGACCTTGTAGAACAGAAGACACAGTTCTTTGACGGTGGAGTAAATGTTG
GAGATACAACAGAACCTGCAACACCTACAACACCTGTAACAACACCGACAACAA
CAGATGATCTGGATGCAGCTAGTGGTCAGTTTAGAGTCATTGGGCCCAGACACCC
TATAAGGGCTCTTGTGGGAGACGAGGTCGAGCTGCCGTGTCGCATTAGTCCAGGC
AAAAACGCCACAGGGATGGAAGTGGGGTGGTACAGGCCTCCCTTCTCTAGGGTT
GTGCATCTCTACCGCAACGGCAAAGATCAGGATGGAGATCAAGCTCCTGAATAT
CGGGGCCGGACTGAGCTGCTCAAGGACGCGATCGGCGAGGGTAAGGTGACCTTG
CGCATCCGAAATGTTAGATTCAGCGATGAAGGCGGATTTACGTGCTTCTTTCGGG
ACCACTCATACCAGGAGGAAGCCGCAATGGAACTGAAGGTGGAGGACCCCTTCT
ATTGGGTATCCCCAGCTAGCTGA
(SEQ ID NO: 22)
LDITSHHHHHHDDLDAVRIKVDTVNAKPGDTVRIPVRFSGIPSKGIANCDFVYSYD
PNVLEIIEIEPGDIIVDPNPDKSFDTAVYPDRKIIVFLFAEDSGTGAYAITKDGVFA
TIVAKVKEGAPNGLSVIKFVEVGGFANNDLVEQKTQFFDGGVNVGDTTEPATPT
TPVTTPTTTDDLDAASGQFRVIGPRHPIRALVGDEVELPCRISPGKNATGMEVGWY
RPPFSRVVHLYRNGKDQDGDQAPEYRGRTELLKDAIGEGKVTLRIRNVRFSDEGGFT
CFFRDHSYQEEAAMELKVEDPFYWVSPAS
6xHis-Cohesin-hMBP
(6xHis underlined; Cohesin in bold; hMBP double underlined)
(SEQ ID NO: 23)
ATGGGCAGCAGCCATCATCATCATCATCACAGCAGCGGCCTGGTGCCGCGCGGC
AGCCATATGGCTAGTATGGATCTGGATGCAGTAAGGATTAAAGTGGACACAGTA
AATGCAAAACCGGGAGACACAGTAAATATACCTGTAAGATTCAGTGGTATACCA
TCCAAGGGAATAGCAAACTGTGACTTTGTATACAGCTATGACCCGAATGTACTTG
AGATAATAGAGATAAAACCGGGAGAATTGATAGTTGACCCGAATCCTACCAAGA
GCTTTGATACTGCAGTATATCCTGACAGAAAGATGATAGTATTCCTGTTTGCGGA
AGACAGCGGAACAGGAGCGTATGCAATAACTAAAGACGGAGTATTTGCTACGAT
AGTAGCGAAAGTAAAAGAAGGAGCACCTAACGGGCTCAGTGTAATCAAATTTGT
AGAAGTAGGCGGATTTGCGAACAATGACCTTGTAGAACAGAAGACACAGTTCTT
TGACGGTGGAGTAAATGTTGGAGATACAACAGAACCTGCAACACCTACAACACC
TGTAACAACACCGACAACAACAGATGATCTAGATGCAGCTAGTGCATCACAAAA
GCGGCCTTCACAACGGCACGGATCTAAATATCTGGCGACAGCCTCTACCATGGAT
CACGCCAGGCATGGCTTTCTGCCCAGGCACAGAGATACTGGAATCTTGGACTCCA
TCGGCAGGTTCTTTGGCGGCGACCGAGGGGCTCCCAAGAGAGGGAGTGGCAAGG
ATAGCCATCATCCAGCCCGAACAGCCCACTACGGAAGCCTGCCGCAGAAAAGCC
ACGGTCGCACGCAGGATGAAAATCCCGTTGTGCACTTCTTCAAAAACATTGTGAC
CCCACGAACTCCTCCACCTTCCCAAGGCAAGGGCAGAGGTCTCAGTCTCAGCCG
GTTCAGTTGGGGGGCCGAGGGCCAGAGACCCGGATTTGGTTATGGGGGAAGGGC
TAGCGACTACAAGTCTGCACATAAGGGGTTCAAAGGGGTCGACGCACAGGGAAC
CCTGTCCAAAATATTTAAGCTTGGTGGCCGCGACTCCCGCTCAGGCTCTCCCATG
GCTCGGCGCTGA
(SEQ ID NO: 24)
MGSSHHHHHHSSGLVPRGSHMASMDLDAVRIKVDTVNAKPGDTVNIPVRFSGIPS
KGIANCDFVYSYDPNVLEIIEIKPGELIVDPNPTKSFDTAVYPDRKMIVFLFAEDS
GTGAYAITKDGVFATIVAKVKEGAPNGLSVIKFVEVGGFANNDLVEQKTQFFDG
GVNVGDTTEPATPTTPVTTPTTTDDLDAASASQKRPSQRHGSKYLATASTMDHAR
HGFLPRHRDTGILDSIGRFFGGDRGAPKRGSGKDSHHPARTAHYGSLPQKSHGRTQD
ENPVVHFFKNIVTPRTPPPSQGKGRGLSLSRESWGAEGQRPGFGYGGRASDYKSAHK
GFKGVDAQGTLSKIFKLGGRDSRSGSPMARR
manti-hASGPR_6.3H9.1D11H-LV-hIgG4H-C-hMOG
(LV-hlgG4H-C underlined, variable region bold underline; hMOG in bold)
(SEQ ID NO: 25)
ATGGAATGGAGCGGGGTCTTTATCTTTCTCCTGTCAGTAACTGCAGGTGCCCACT
CCCAGGTCCAGCTGCAGCAGTCTGGAGCTGAGCTGGTAAGGCCTGGGACTTCAG
TGAAGATGTCCTGCGAGGCTGCTAGATTCACCTTCAGTAACTACTGGATTGGTTG
GGTAAAGCAGAGGCCTGGACATGGCCTTGAGTGGATTGGAGATATTTTCCCTGG
AGGTGATTATACTAACTACAATAAGAAATTCAAGGACAAGGCCACACTGACTGC
AGACACATCCTCCAGCACAGCCTACATGCAGCTCAGCAGCCTGACATCTGAGGA
CTCTGCCATCTATTACTGTGCAAGATCGGACTACGGTGGTTACTACGTCTTTGACT
ACTGGGGCCAAGGCACCACTCTCACAGTCTCCTCAGCCAAAACAAAGGGCCCAT
CCGTCTTCCCCCTGGCGCCCTGCTCCAGGAGCACCTCCGAGAGCACAGCCGCCCT
GGGCTGCCTGGTCAAGGACTACTTCCCCGAACCGGTGACGGTGTCGTGGAACTCA
GGCGCCCTGACCAGCGGCGTGCACACCTTCCCGGCTGTCCTACAGTCCTCAGGAC
TCTACTCCCTCAGCAGCGTGGTGACCGTGCCCTCCAGCAGCTTGGGCACGAAGAC
CTACACCTGCAACGTAGATCACAAGCCCAGCAACACCAAGGTGGACAAGAGAGT
TGAGTCCAAATATGGTCCCCCATGCCCACCCTGCCCAGCACCTGAGTTCGAAGGG
GGACCATCAGTCTTCCTGTTCCCCCCAAAACCCAAGGACACTCTCATGATCTCCC
GGACCCCTGAGGTCACGTGCGTGGTGGTGGACGTGAGCCAGGAAGACCCCGAGG
TCCAGTTCAACTGGTACGTGGATGGCGTGGAGGTGCATAATGCCAAGACAAAGC
CGCGGGAGGAGCAGTTCAACAGCACGTACCGTGTGGTCAGCGTCCTCACCGTCC
TGCACCAGGACTGGCTGAACGGCAAGGAGTACAAGTGCAAGGTCTCCAACAAAG
GCCTCCCGTCCTCCATCGAGAAAACCATCTCCAAAGCCAAAGGGCAGCCCCGAG
AGCCACAGGTGTACACCCTGCCCCCATCCCAGGAGGAGATGACCAAGAACCAGG
TCAGCCTGACCTGCCTGGTCAAAGGCTTCTACCCCAGCGACATCGCCGTGGAGTG
GGAGAGCAATGGGCAGCCGGAGAACAACTACAAGACCACGCCTCCCGTGCTGGA
CTCCGACGGCTCCTTCTTCCTCTACAGCAGGCTAACCGTGGACAAGAGCAGGTGG
CAGGAGGGGAATGTCTTCTCATGCTCCGTGATGCATGAGGCTCTGCACAACCACT
ACACACAGAAGAGCCTCTCCCTGTCTCTGGGTAAAGCTAGTGGTCAGTTTAGAGT
CATTGGGCCCAGACACCCTATAAGGGCTCTTGTGGGAGACGAGGTCGAGCTGCC
GTGTCGCATTAGTCCAGGCAAAAACGCCACAGGGATGGAAGTGGGGTGGTACAG
GCCTCCCTTCTCTAGGGTTGTGCATCTCTACCGCAACGGCAAAGATCAGGATGGA
GATCAAGCTCCTGAATATCGGGGCCGGACTGAGCTGCTCAAGGACGCGATCGGC
GAGGGTAAGGTGACCTTGCGCATCCGAAATGTTAGATTCAGCGATGAAGGCGGA
TTTACGTGCTTCTTTCGGGACCACTCATACCAGGAGGAAGCCGCAATGGAACTGA
AGGTGGAGGACCCCTTCTATTGGGTATCCCCAGCTAGCTGA
(SEQ ID NO: 26)
QVQLQQSGAELVRPGTSVKMSCEAARFTFSNYWIGWVKQRPGHGLEWIGDIFP
GGDYTNYNKKFKDKATLTADTSSSTAYMQLSSLTSEDSAIYYCARSDYGGYYVF
DYWGQGTTLTVSSAKTKGPSVFPLAPCSRSTSESTAALGCLVKDYFPEPVTVSWNS
GALTSGVHTFPAVLQSSGLYSLSSVVTVPSSSLGTKTYTCNVDHKPSNTKVDKRVES
KYGPPCPPCPAPEFEGGPSVFLFPPKPKDTLMISRTPEVTCVVVDVSQEDPEVQFNWY
VDGVEVHNAKTKPREEQFNSTYRVVSVLTVLHQDWLNGKEYKCKVSNKGLPSSIEK
TISKAKGQPREPQVYTLPPSQEEMTKNQVSLTCLVKGFYPSDIAVEWESNGQPENNY
KTTPPVLDSDGSFFLYSRLTVDKSRWQEGNVFSCSVMHEALHNHYTQKSLSLSLGK
ASGQFRVIGPRHPIRALVGDEVELPCRISPGKNATGMEVGWYRPPFSRVVHLYR
NGKDQDGDQAPEYRGRTELLKDAIGEGKVTLRIRNVRFSDEGGFTCFFRDHSY
QEEAAMELKVEDPFYWVSPAS
manti-hASGPR_6.3H9.1D11K-LV-hIgGK-C
(LV-hIgGK-C underlined, variable region bold underline)
(SEQ ID NO: 27)
ATGGATTCACAGGCCCAGGTTCTTATGTTACTGCTGCTATGGGTATCTGGTACCT
GTGGGGACATTGTGATGTCACAGTCTCCATCCTCCCTAGCTGTGTCAGTTGGAGA
GAAGGTTACTATGAGCTGCAAGTCCAGTCAGAACCTTTTATATAGTAGCAATCAA
AAGAACTACTTGGCCTGGTACCAGCAGAAACCAGGGCAGTCTCCTAAACTGCTG
ATTTACTGGGCATCCACTAGGGAATCTGGGGTCCCTGATCGCTTCACAGGCAGTG
GATCTGGGACAGATTTCACTCTCACCATCAGCAGTGTGAAGGCTGAAGACCTGGC
AGTCTATTACTGTCAGCAATATTATAGCTATCCTTACACGTTCGGAGGGGGGACC
AAGCTCGAGATCAAACGAACTGTGGCTGCACCATCTGTCTTCATCTTCCCGCCAT
CTGATGAGCAGTTGAAATCTGGAACTGCCTCTGTTGTGTGCCTGCTGAATAACTT
CTATCCCAGAGAGGCCAAAGTACAGTGGAAGGTGGATAACGCCCTCCAATCGGG
TAACTCCCAGGAGAGTGTCACAGAGCAGGACAGCAAGGACAGCACCTACAGCCT
CAGCAGCACCCTGACGCTGAGCAAAGCAGACTACGAGAAACACAAAGTCTATGC
CTGCGAAGTCACCCATCAGGGCCTGAGCTCGCCCGTCACAAAGAGCTTCAACAG
GGGAGAGTGTGCTAGCTGA
(SEQ ID NO: 28)
DIVMSQSPSSLAVSVGEKVTMSCKSSQNLLYSSNQKNYLAWYQQKPGQSPKLLI
YWASTRESGVPDRFTGSGSGTDFTLTISSVKAEDLAVYYCQQYYSYPYTFGGGT
KLEIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGECAS
manti-hASGPR_5H8.1D4H-LV-hIgG4H-C-hMOG
(LV-hlgG4H-C underlined, variable region bold underline; hMOG in bold)
(SEQ ID NO: 29)
ATGGCTTGGGTGTGGACCTTGCTATTCCTGATGGCAGCCGCCCAAAGTATCCAAG
CACAGATCCAGTTGGTGCAGTCTGGACCTGAGCTGAAGAAGCCTGGAGAGACAG
TCAAGATCTCCTGCAAGGCTTCTGGTTATACCTTCACAGACTATTCAGTGCACTG
GGTGAAGCAGGCTCCAGGAAAGGGTTTAAAGTGGATGGGCTGGATAAATACTGA
GACTGGTGAGCCAACATATGCAGATGACCTCAAGGGACGGTTTGCCTTCTCTTTG
GAAACCTCTGCCAGCACTGCCTATTTGCAGATCAACAACCTCAAAAATGAGGAC
ACGGCTACATATTTCTGTGCTAAACCTACCTATAGATTTTTTGACTACTGGGGCCA
AGGCACCACTCTCACAGCCTCCTCAGCCAAAACGAAGGGCCCATCCGTCTTCCCC
CTGGCGCCCTGCTCCAGGAGCACCTCCGAGAGCACAGCCGCCCTGGGCTGCCTG
GTCAAGGACTACTTCCCCGAACCGGTGACGGTGTCGTGGAACTCAGGCGCCCTG
ACCAGCGGCGTGCACACCTTCCCGGCTGTCCTACAGTCCTCAGGACTCTACTCCC
TCAGCAGCGTGGTGACCGTGCCCTCCAGCAGCTTGGGCACGAAGACCTACACCT
GCAACGTAGATCACAAGCCCAGCAACACCAAGGTGGACAAGAGAGTTGAGTCCA
AATATGGTCCCCCATGCCCACCCTGCCCAGCACCTGAGTTCGAAGGGGGACCATC
AGTCTTCCTGTTCCCCCCAAAACCCAAGGACACTCTCATGATCTCCCGGACCCCT
GAGGTCACGTGCGTGGTGGTGGACGTGAGCCAGGAAGACCCCGAGGTCCAGTTC
AACTGGTACGTGGATGGCGTGGAGGTGCATAATGCCAAGACAAAGCCGCGGGAG
GAGCAGTTCAACAGCACGTACCGTGTGGTCAGCGTCCTCACCGTCCTGCACCAGG
ACTGGCTGAACGGCAAGGAGTACAAGTGCAAGGTCTCCAACAAAGGCCTCCCGT
CCTCCATCGAGAAAACCATCTCCAAAGCCAAAGGGCAGCCCCGAGAGCCACAGG
TGTACACCCTGCCCCCATCCCAGGAGGAGATGACCAAGAACCAGGTCAGCCTGA
CCTGCCTGGTCAAAGGCTTCTACCCCAGCGACATCGCCGTGGAGTGGGAGAGCA
ATGGGCAGCCGGAGAACAACTACAAGACCACGCCTCCCGTGCTGGACTCCGACG
GCTCCTTCTTCCTCTACAGCAGGCTAACCGTGGACAAGAGCAGGTGGCAGGAGG
GGAATGTCTTCTCATGCTCCGTGATGCATGAGGCTCTGCACAACCACTACACACA
GAAGAGCCTCTCCCTGTCTCTGGGTAAAGCTAGTGGTCAGTTTAGAGTCATTGGG
CCCAGACACCCTATAAGGGCTCTTGTGGGAGACGAGGTCGAGCTGCCGTGTCGC
ATTAGTCCAGGCAAAAACGCCACAGGGATGGAAGTGGGGTGGTACAGGCCTCCC
TTCTCTAGGGTTGTGCATCTCTACCGCAACGGCAAAGATCAGGATGGAGATCAAG
CTCCTGAATATCGGGGCCGGACTGAGCTGCTCAAGGACGCGATCGGCGAGGGTA
AGGTGACCTTGCGCATCCGAAATGTTAGATTCAGCGATGAAGGCGGATTTACGTG
CTTCTTTCGGGACCACTCATACCAGGAGGAAGCCGCAATGGAACTGAAGGTGGA
GGACCCCTTCTATTGGGTATCCCCAGCTAGCTGA
(SEQ ID NO: 30)
AQIQLVQSGPELKKPGETVKISCKASGYTFTDYSVHWVKQAPGKGLKWMGWI
NTETGEPTYADDLKGRFAFSLETSASTAYLQINNLKNEDTATYFCAKPTYRFFDY
WGQGTTLTASSAKTKGPSVFPLAPCSRSTSESTAALGCLVKDYFPEPVTVSWNSGA
LTSGVHTFPAVLQSSGLYSLSSVVTVPSSSLGTKTYTCNVDHKPSNTKVDKRVESKY
GPPCPPCPAPEFEGGPSVFLFPPKPKDTLMISRTPEVTCVVVDVSQEDPEVQFNWYVD
GVEVHNAKTKPREEQFNSTYRVVSVLTVLHQDWLNGKEYKCKVSNKGLPSSIEKTIS
KAKGQPREPQVYTLPPSQEEMTKNQVSLTCLVKGFYPSDIAVEWESNGQPENNYKT
TPPVLDSDGSFFLYSRLTVDKSRWQEGNVFSCSVMHEALHNHYTQKSLSLSLGKAS
GQFRVIGPRHPIRALVGDEVELPCRISPGKNATGMEVGWYRPPFSRVVHLYRNG
KDQDGDQAPEYRGRTELLKDAIGEGKVTLRIRNVRFSDEGGFTCFFRDHSYQEE
AAMELKVEDPFYWVSPAS
manti-hASGPR_5H8.1D4K-LV-hIgGK-C
(LV-hIgGK-C underlined, variable region bold underline)
(SEQ ID NO: 31)
ATGGATTCACAGGCCCAGGTTCTTATATTGCTGCTGCTATGGGTATCTGGTTCCTG
TGGGGACATTGTGATGTCACAGTCTCCATCCTCCCTGGCTGTGTCAGCAGGAGAG
AAGGTCACTATGAGCTGCAAATCCAGTCAGAGTCTGCTCAACAGTAGAACCCGA
AAGAACTACTTGGCTTGGTACCAGCAGAAACCAGGGCAGTCTCCTAAACTGCTG
ATCTACTGGGCATCCACTAGGGAATCTGGGGTCCCTGATCGCTTCACAGGCAGTG
GATCTGGGACAGATTTCACTCTCACCATCAGCAGTGTGCAGGCTGAGGACCTGGC
AGTTTATTACTGCAAGCAATCTTATAATCTGTGGACGTTCGGTGGAGGCACCAAG
CTCGAGATCAAACGAACTGTGGCTGCACCATCTGTCTTCATCTTCCCGCCATCTG
ATGAGCAGTTGAAATCTGGAACTGCCTCTGTTGTGTGCCTGCTGAATAACTTCTA
TCCCAGAGAGGCCAAAGTACAGTGGAAGGTGGATAACGCCCTCCAATCGGGTAA
CTCCCAGGAGAGTGTCACAGAGCAGGACAGCAAGGACAGCACCTACAGCCTCAG
CAGCACCCTGACGCTGAGCAAAGCAGACTACGAGAAACACAAAGTCTATGCCTG
CGAAGTCACCCATCAGGGCCTGAGCTCGCCCGTCACAAAGAGCTTCAACAGGGG
AGAGTGTGCTAGCTGA
(SEQ ID NO: 32)
DIVMSQSPSSLAVSAGEKVTMSCKSSQSLLNSRTRKNYLAWYQQKPGQSPKLLI
YWASTRESGVPDRFTGSGSGTDFTLTISSVQAEDLAVYYCKQSYNLWTFGGGTK
LEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGECAS

### Modular Domain Description of DC-Targeting Multiple Sclerosis Immunotherapeutics

In certain aspects a DC-targeting multiple sclerosis immunotherapeutic may be assembled by combining polypeptides domains belonging to various classes of proteins categorized according to a specific function. In a general sense these domains may belong to classes comprising antibodies, antibody CDRs, antibody heavy chains, antibody light chains, linkers, antigens, coupling domains, adjuvants, purification tags, labelling tags or reporter tags.

Non limiting examples of domain categories and specific examples within each category are illustrated in Table 1. (FlgIn is abbreviation for Flagellin)

| Peptide | SEQ | | SEQ | Coupling | SEQ |
|---|---|---|---|---|---|
| Linkers | ID NO | Antigens | ID NO | Domains | ID NO |
| Flex-v1 | 33 | hMBP | 36 | Cohesin | 38 |
| Flexx-v1 | 34 | hMOG | 37 | Dockerin | 39 |
| Flexx-v2 | 35 | | | Dockerinv2 | 40 |
| | SEQ | | | | SEQ |
| Adjuvants | ID NO | Antibodies | | | ID NO |
| hIL-10 | 41 | mAnti-ASGPR_49C11_7H (heavy chain) | | | 42 |
| | | mAnti-ASGPR_49C11_7K (light chain) | | | 43 |
| | | manti-hASGPR_6.3H9.1D11H (heavy chain) | | | 44 |
| | | manti-hASGPR_6.3H9.1D11K (light chain) | | | 45 |
| | | manti-hASGPR_5H8.1D4H (heavy chain) | | | 46 |
| | | manti-hASGPR_5H8.1D4K (light chain) | | | 47 |
| | | mAnti-ASGPR_4G2.2_(heavy chain) | | | 48 |
| | | mAnti-ASGPR_4G2.2_(light chain) | | | 49 |
| | | mAnti-ASGPR-5F10H(heavy chain) | | | 50 |
| | | mAnti-ASGPR-5F10H(light chain) | | | 51 |
| | | mAnti-ASGPR1H11(heavy chain) | | | 52 |
| | | mAnti-ASGPR1H11(light chain) | | | 53 |

In some embodiments, components of a DC-targeting immunotherapeutic may be constructed as illustrated below (For the schematic representations that follow, the following abbreviations apply: Peptide Linker (PL); Antigen (Ag); Tag (Tg); Coupling Domain (CD); Adjuvant (Adj); Antibody (Ab). A number following an abbreviation differentiates between different types of that domain within a construct. A hyphen (“-”) used may represent a covalent bond, such as a peptide bond between two domains of a polypeptide that is formed during translation of, for example, a fusion protein. The covalent bond may also be formed by, but is not limited to, known chemical coupling means. A hyphen may also represent a high-affinity, intermediate affinity, or low affinity non-covalent interaction. Examples of these types of non-covalent interactions are known to those skilled in the art and include, but are not limited to, antibody/antigen interaction, receptor/ligand interaction, avidin/biotin interaction, cohesin/dockerin interaction and barnase/barstar interaction.):

CD-Ag-Tg;

Ab-Ag-Tg;

Ab-CD-Ag-Tg;

Ab-PL-Ag;

Ab-PL-Ag-Tg;

Ab-PL-Ag(1)-Ag(2)-Tg;

Ab-CD-PL;

Ab-Ag;

Tg-CD-Ag;

Tg-CD-Ag-Tg;

Ab-Adj;

Ab-Adj-Adj;

Tg-CD-Adj;

Tg-CD-Adj(1)-Adj(2);

CD-Adj;

Ab-PL-Ag-PL-Ag;
PL includes but is not limited to peptide linkers. Linkers with non-peptide bonds are also contemplated. In some embodiments the tag is absent from the construct or has been removed.

In one particular embodiment, an antibody-antigen fusion protein (Ab.Ag) comprises the following formula:

Ab-(PL-Ag)x;

Ab-(Ag-PL)x;

Ab-(PL-Ag-PL)x;

Ab-(Ag-PL-Ag)x;

Ab-(PL-Ag)x-PL; or

Ab-(Ag-PL)x-Ag;
wherein Ab is an DC targeting antibody or a fragment thereof; wherein PL is a peptide linker; wherein Ag is an myelin sheath protein or component; and, wherein x is an integer from 1 to 20, or any range derivable therein. PL includes but is not limited to peptide linkers. Linkers with non-peptide bonds are also contemplated.

In one embodiment, the -(PL-Ag) x, -(Ag-PL)x, -(PL-Ag-PL)x, or -(Ag-PL-Ag)x are located at the carboxy terminus of the Ab heavy chain or fragment thereof.

In another embodiment, the -(PL-Ag)x, -(Ag-PL)x, -(PL-Ag-PL)x, or -(Ag-PL-Ag)x are located at the carboxy terminus of the Ab light chain or fragment thereof.

In one embodiment, the antibody-antigen complex (Ab:Ag) comprises the following formula

Ab.Doc:Coh.Ag;

Ab.Coh:Doc.Ag;

Ab.(Coh)x:(Doc.Ag)x;

Ab.(Doc)x:(Coh.Ag)x;

Ab.(Coh.Doc)x:(Doc.Ag¹)(Coh.Ag²); or

Ab.(Coh)x(Doc)x:(Doc.Ag¹)x(Coh.Ag²)x;
wherein Ab is a DC targeting antibody or a fragment thereof; wherein Ag is an myelin sheath protein or component (Ag¹ and Ag² being two distinct myelin sheath protein or components); wherein Doc is dockerin; wherein Coh is cohesin and wherein x is an integer from 1 to 10, or any range derivable therein, denoting the number of molecules or domains in the parentheses immediately preceding it. A period (“.”) is used to denote a covalent bond between two molecules or domains (examples of these covalent bonds include, but are not limited to, a peptide bond between two domains of a polypeptide that is formed during translation of, for example, a fusion protein. The covalent bond may also be formed by, but is not limited to, known chemical coupling means). A colon (“:”) is used to denote a non-covalent interaction between a cohesin and dockerin domain.

### IV. METHODS OF TREATMENT

As discussed above, the compositions and methods of using these compositions can treat a subject (e.g., prevent multiple sclerosis or evoke a robust immune tolerance to a multiple sclerosis autoimmune bout) having, suspected of having, or at risk of developing an autoimmune disorder or related disease, particularly those related to multiple sclerosis.

As used herein the phrase “immune response” or its equivalent “immunological response” refers to a humoral (antibody mediated), cellular (mediated by antigen-specific T cells or their secretion products) or both humoral and cellular response directed against a protein, peptide, or polypeptide of the embodiments in a recipient patient. Treatment or therapy can be an active immune response induced by administration of immunogen or a passive therapy effected by administration of antibody, antibody containing material, or primed T-cells.

For purposes of this specification and the accompanying claims the terms “epitope” and “antigenic determinant” are used interchangeably to refer to a site on an antigen to which B and/or T cells respond or recognize. B-cell epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include those methods described in Epitope Mapping Protocols (1996). T cells recognize continuous epitopes of about nine amino acids for CD8 cells or about 13-15 amino acids for CD4 cells. T cells that recognize the epitope can be identified by in vitro assays that measure antigen-dependent proliferation, as determined by 3H-thymidine incorporation by primed T cells in response to an epitope (Burke et al., 1994), by antigen-dependent killing (cytotoxic T lymphocyte assay, Tigges et al., 1996) or by cytokine secretion.

The presence of a cell-mediated immunological response can be determined by proliferation assays (CD4(+) T cells) or CTL (cytotoxic T lymphocyte) assays. The relative contributions of humoral and cellular responses to the protective or therapeutic effect of an immunogen can be distinguished by separately isolating IgG and T-cells from an immunized syngeneic animal and measuring protective or therapeutic effect in a second subject. As used herein and in the claims, the terms “antibody” or “immunoglobulin” are used interchangeably.

Optionally, an antibody or preferably an immunological portion of an antibody, can be chemically conjugated to, or expressed as, a fusion protein with other proteins. For purposes of this specification and the accompanying claims, all such fused proteins are included in the definition of antibodies or an immunological portion of an antibody.

In one embodiment a method includes treatment for a disease or condition caused by an autoimmune disorder. In certain aspects embodiments include methods of treatment of multiple sclerosis. In some embodiments, the treatment is administered in the presence of myelin sheath protein or components. Furthermore, in some examples, treatment comprises administration of other agents commonly used against autoimmune disorders, such as one or more immunosuppressant compounds.

The therapeutic compositions are administered in a manner compatible with the dosage formulation, and in such amount as will be therapeutically effective. The quantity to be administered depends on the subject to be treated. Precise amounts of active ingredient required to be administered depend on the judgment of the practitioner. Suitable regimes for initial administration and boosters are also variable, but are typified by an initial administration followed by subsequent administrations.

Compositions of the current methods may be administered to patients via any route used to introduce vaccines or antibodies to patients. Such routes include, but are not limited to, mucosal or intramuscular delivery. In particular embodiments, a composition is administered to a patient intranasally or by inhalation. In other embodiments, a composition is administered intravenously or by intravenous injection. In additional embodiments, the administration of compositions includes, but is not limited to oral, parenteral, subcutaneous, intramuscular, intravenous administration, or various combinations thereof.

The manner of application may be varied widely. Any of the conventional methods for administration of a polypeptide therapeutic are applicable. These are believed to include oral application on a solid physiologically acceptable base or in a physiologically acceptable dispersion, parenterally, by injection and the like. The dosage of the composition will depend on the route of administration and will vary according to the size and health of the subject. In one treatment scheme, the patient receives a subcutaneous dose of the immunotherapeutic every week for three weeks and then every first week for an additional 1, 2, 3, 4, 5, 6, 7, 8, 9, 10, 11, or 12 months.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9,10, 11, 12 twelve week intervals, including all ranges there between.

### Combination Therapy

The compositions and related methods, particularly administration of an antibody that binds DC receptor and delivers a myelin sheath protein or component or a peptide to a patient/subject, may also be used in combination with the administration of multiple sclerosis effective strategies or traditional immunomodulatory therapies. Such strategies or therapies may be directed, among other aims, to modify the disease course, treat exacerbations, manage symptoms or improve a compromised function. Examples of disease-modifying agents include, but are not limited to, Aubagio (teriflunomide), Avonex (interferon beta-1a), Betaseron and Extavia (interferon beta-1b), Copaxone (glatiramer acetate), Extavia (interferon beta-1b), Gilenya (fingolimod), Novantrone (mitoxantrone), Rebif (interferon beta-1a), Tecfidera (dimethyl fumarate), and Tysabri (natalizumab). In other embodiments the disease-modifying therapeutic to be used in combination therapy include, but are not limited to, Fingolimod (Gilenya), Methotrexate, azathioprine (Imuran), intravenous immunoglobulin (IVIg) and cyclophosphamide (Cytoxan).

In some instances the combination therapeutic may be used to control symptoms. Examples of medications or pharmaceuticals that may be used to control multiple sclerosis symptoms include, but are not limited to, dalfamipridine (Ampyra), tizanidine (Zanaflex), diazepam (Valium), clonazepam (Klonopin), dantrolene (Dantrium), baclofen (Lioresal), or any benzodiazepine, cholinergic medications, or amantadine.

In one aspect, it is contemplated that a therapy is used in conjunction with immunosuppressants. In other aspects, a therapy is used in conjunction with disease-modifying agents, symptom controlling agents, or agents to improve compromised function. Alternatively, the therapy may precede or follow the other agent treatment by intervals ranging from minutes to weeks. In embodiments where the other agents and/or a proteins or polynucleotides are administered separately, one would generally ensure that a significant period of time did not expire between the time of each delivery, such that the therapeutic composition would still be able to exert an advantageously combined effect on the subject. In such instances, it is contemplated that one may administer both modalities within about 12-24 h of each other and, more preferably, within about 6-12 h of each other. In some situations, it may be desirable to extend the time period for administration significantly, however, where several days (2, 3, 4, 5, 6 or 7) to several weeks (1, 2, 3, 4, 5, 6, 7 or 8) lapse between the respective administrations.

Various combinations of therapy may be employed, for example immunosuppressant therapy, disease-modifying agents, symptom controlling agents, or agents to improve compromised function is “A” and an antibody immunotherapeutic that comprises an antibody that binds a DC receptor and delivers an myelin sheath protein or component or a peptide or consensus peptide thereof is “B”:
A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the antibody compositions to a patient/subject will follow general protocols for the administration of such compounds, taking into account the toxicity, if any, of the composition. It is expected that the treatment cycles would be repeated as necessary. It is also contemplated that various standard therapies, such as hydration, may be applied in combination with the described therapy.

### General Pharmaceutical Compositions

In some embodiments, pharmaceutical compositions are administered to a subject. Different aspects may involve administering an effective amount of a composition to a subject. In some embodiments, an antibody that binds DC receptor and delivers a myelin sheath protein or component or a peptide or consensus peptide thereof may be administered to the patient to protect against or treat against multiple sclerosis. Alternatively, an expression vector encoding one or more such antibodies or polypeptides or peptides may be given to a patient as a preventative treatment. Additionally, such compositions can be administered in combination with an immunosuppressant. Such compositions will generally be dissolved or dispersed in a pharmaceutically acceptable carrier or aqueous medium.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents, immunosuppressants and immunotherapeutics, can also be incorporated into the compositions.

The active compounds can be formulated for parenteral administration, e.g., formulated for injection via the intravenous, intramuscular, sub-cutaneous, or even intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaccous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, nasal, or buccal administration. Alternatively, administration may be by orthotopic, intradermal, subcutaneous, intramuscular, intraperitoneal, intranasal, or intravenous injection. In certain embodiments, a immunotherapeutic composition may be inhaled (e.g., U.S. Pat. No. 6,651,655, which is specifically incorporated by reference). Such compositions would normally be administered as pharmaceutically acceptable compositions that include physiologically acceptable carriers, buffers or other excipients.

An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the protection desired.

Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

Upon formulation, solutions will be administered in a manner compatible with the dosage formulation and in such amount as is therapeutically or prophylactically effective. The formulations are easily administered in a variety of dosage forms, such as the type of injectable solutions described above.

### IV. EXAMPLES

The following examples are included to demonstrate preferred embodiments. It should be appreciated by those of skill in the art that the techniques disclosed in the examples which follow represent techniques discovered by the inventors to function well in the practice of the embodiments, and thus can be considered to constitute preferred modes for their practice. However, those of skill in the art should, in light of the present disclosure, appreciate that many changes can be made in the specific embodiments which are disclosed and still obtain a like or similar result without departing from the spirit and scope of the invention.

### Example 1—Characterization and Response to Anti-DC-ASGPR Antibodies

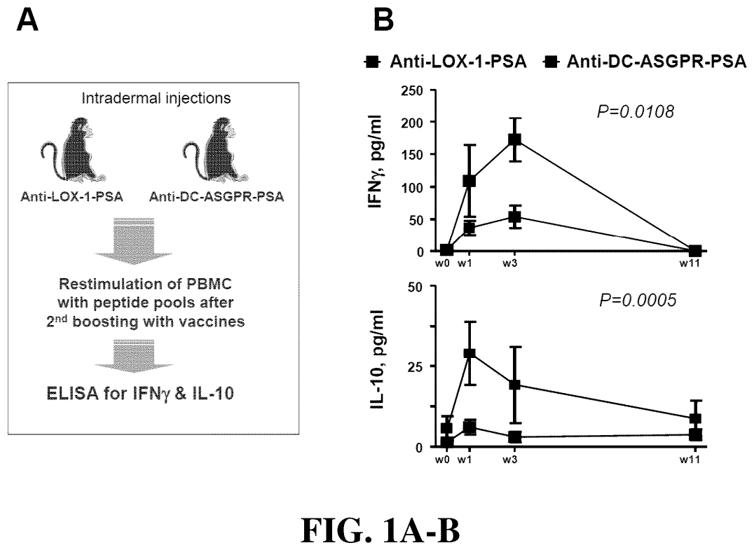

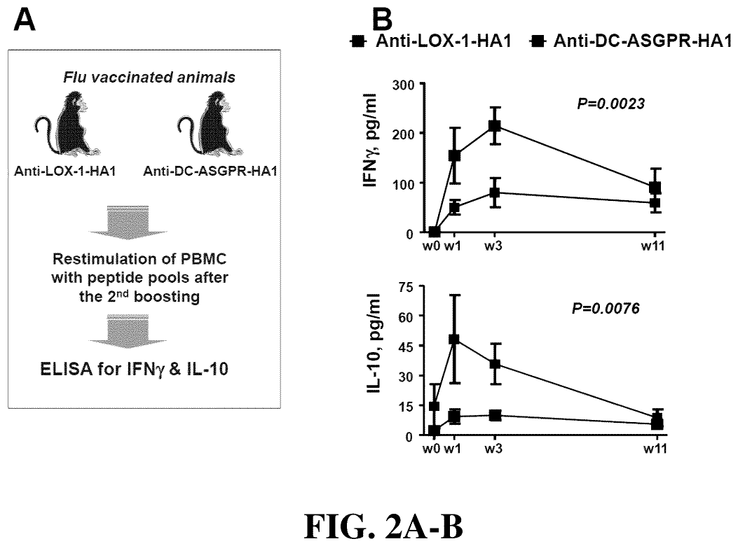

All animals (total 12 animals: 6 animals per group) were pre-immunized with live influenza viruses (H1N1, PR8). Sera from all animals displayed HA1-specific IgG (data not shown). Four months after priming, animals were immunized i.d. with either anti-LOX-1-HA1 (right arm) and anti-LOX-1-PSA (left arm) or anti-DC-ASGPR-HA1 (right arm) and anti-DC-ASGPR-PSA (left arm). After three immunizations at 40 days intervals with the same recombinant fusion proteins, blood was collected as indicated. PBMCs from animals immunized with anti-DC-ASGPR-HA1 secreted higher levels of IL-10 in response to HAI peptide pool when compared to those immunized with anti-LOX-1-HA1 (FIG. 2B, upper panel). Conversely, PBMCs from animals immunized with anti-LOX-1-HA1 secreted significantly higher levels of IFNγ than animals immunized with anti-DC-ASGPR-HA1 (FIG. 2B, lower panel). The same findings were made with animals that were primed and boosted twice with PSA fusion proteins. PSA-specific IL-10-producing cellular responses were preferentially mounted in animals immunized with anti-DC-ASGPR-PSA (FIG. 1B, upper panel). Animals immunized with anti-LOX-1-PSA mounted higher PSA-specific IFNγ-producing cellular responses than animals immunized with anti-DC-ASGPR-PSA (FIG. 1B, lower panel). For both HA1 and PSA, the peak of IL-10-producing cellular responses was obtained at week one, but the peak of IFNγ-producing cellular responses was obtained at week three. Taken together, the data show that targeting antigens to in vivo DCs via DC-ASGPR can establish antigen-specific IL-10-producing T cells in vivo.

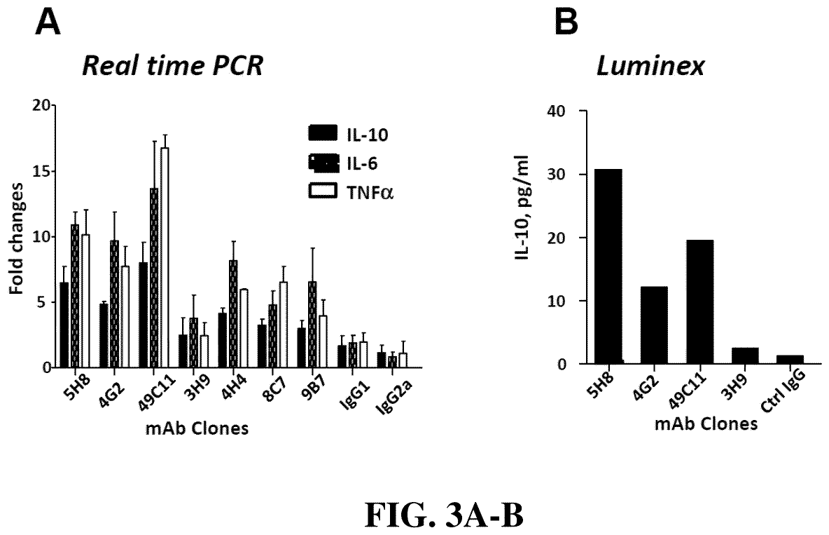

Monocyte-derived IFNDCs were cultured overnight in the plates coated with indicated monoclonal antibodies. Cells were harvested and RNA expression levels of IL-10, IL-6 and TNFalpha was assessed by real time PCR using commercially available PCR primers. Compared to other clones of anti-DC-ASGPR antibodies, 5H8 and 49C11 resulted in increased expression of IL-10. They also induced increased levels of IL-6 and TNFa (FIG. 3A).

The amount of IL-10 in the culture supernatants were assessed by Luminex assay. Consistent with the data in the left panel, 5H8 and 49C11 induced IFNDCs to secrete increased amount of IL-10 (FIG. 3B).

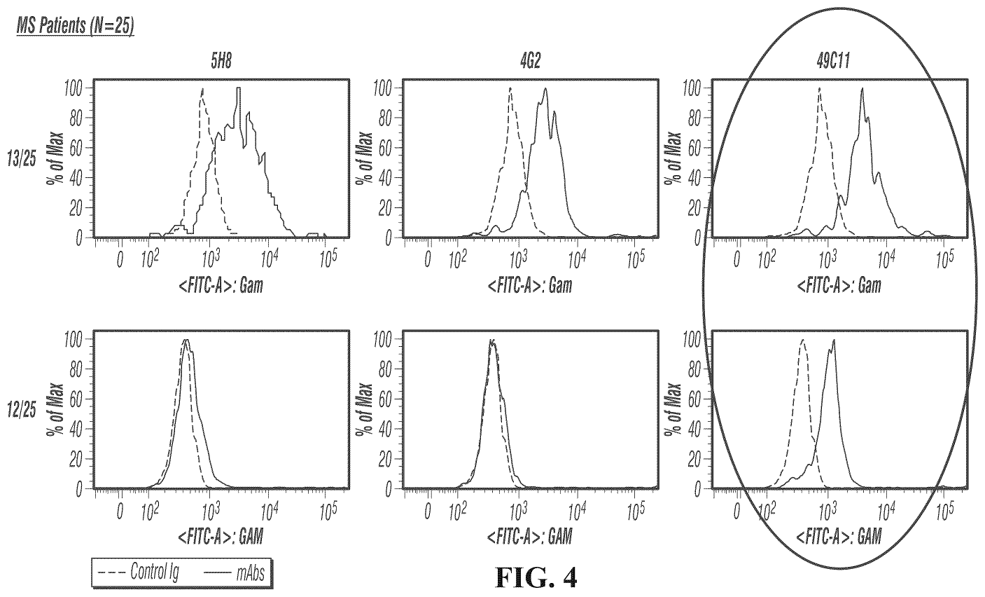

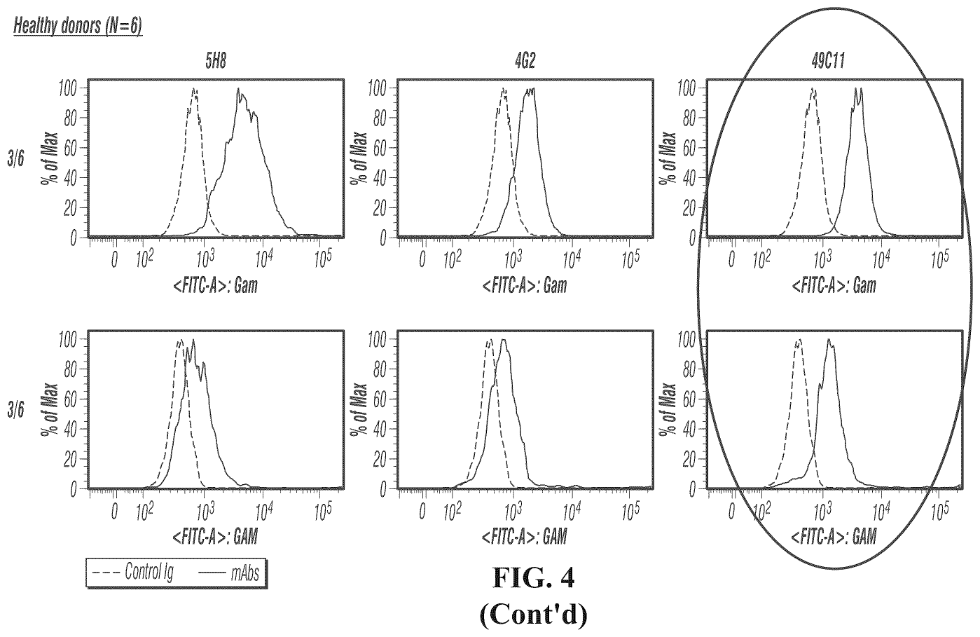

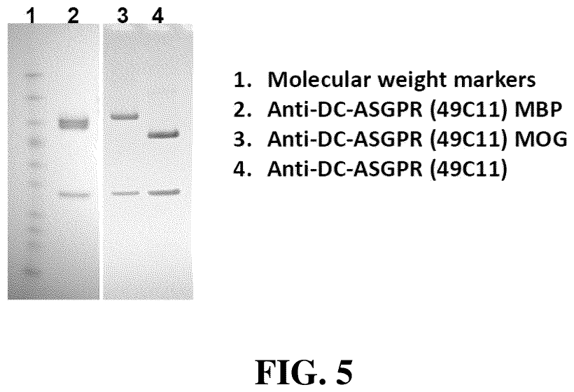

CD11c+ blood DCs from healthy donors (n=6) and MS patients (n=25) were stained with 5H8, 4G2 and 49C11. CD11c+ DCs from both healthy and patient donors displayed two distinct patterns of anti-DC-ASGPR antibody bindings: all of the anti-DC-ASGPR antibodies bound well to CD11c+ DCs from approximately 50% of the donors, while CD11c+ DCs from the other 50% of donors were weakly stained with the three clones of anti-DC-ASGPR antibodies. However, 49C11 was able to bind to CD11c+ DCs better than the other two clones. In addition to its (49C11) ability to induce IL-10, 49C11 can bind well to DCs. Thus 49C11 was selected as a clone to be fused to MS antigens (FIG. 4).

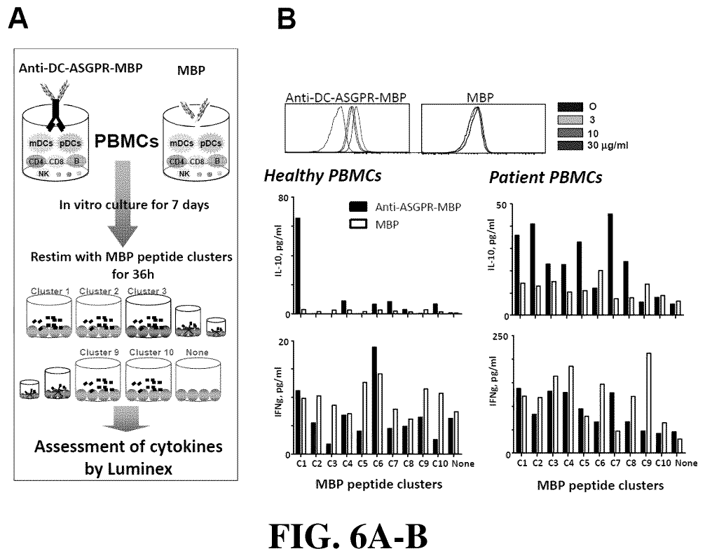

CD11c+ DCs from healthy donors were stained with different concentrations (0, 3, 10, and 30 ug/ml) of anti-DC-ASGPR-MBP or MBP alone. The data indicate that anti-DC-ASGPR-MBP binds well to the DCs (FIG. 6B, upper panels).

PBMCs from healthy and MS patient donors were loaded with 5 ug/ml anti-DC-ASGPR-MBP fusion protein or MBP alone. Cells were incubated for 7 days and then T cells were restimulated for 48 h in the presence of MBP-derived peptide clusters. IFNg and IL-10 secreted from T cells were assessed by the Luminex. Compared to MBP, anti-DC-ASGPR-MBP resulted in increased MBP-specific IL-10 producing T cell responses in both healthy and patient donors (FIG. 6B, lower panels).

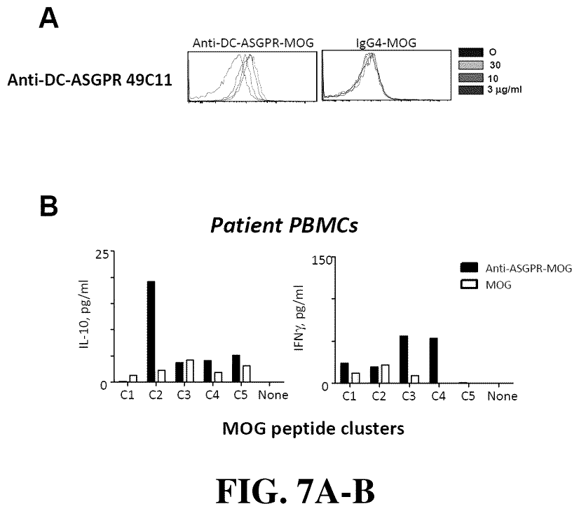

CD11c+ DCs from healthy donors were stained with different concentrations (0, 3, 10, and 30 ug/ml) of anti-DC-ASGPR-MOG or MOG alone. The data indicate that anti-DC-ASGPR-MOG binds well to the DCs (FIG. 7A).

(Lower panels) PBMCs from MS patient donor were loaded with 5 ug/ml anti-DC-ASGPR-MOG fusion protein or MBP alone. Cells were incubated for 7 days and then T cells were restimulated for 48 h in the presence of MBP-derived peptide clusters. IFNg and IL-10 secreted from T cells were assessed by the Luminex. Compared to MOG, anti-DC-ASGPR-MOG resulted in increased MBP-specific IL-10 producing T cell responses in both healthy and patient donors (FIG. 7B).

### Example 2—Effect of Anti-DC-ASGPR-MOG on EAE Induction/Progression in NHP

The following experimental results were kindly provided by Dr. Roger Le Grand, who worked with the inventors.

To test the effects of anti-DC-ASGPR-MOG on the development/progression of EAE in non-human primates (NHP), an EAE model in cynomolgus macaques was used.

Experimental autoimmune encephalomyelitis (EAE), sometimes referred to as experimental allergic encephalomyelitis, is an animal model of brain inflammation. It is an inflammatory demyelinating disease of the central nervous system (CNS). Originally used with rodents, it is widely studied as an animal model of human CNS demyelinating diseases, including multiple sclerosis and acute disseminated encephalomyelitis (ADEM). EAE is also the prototype for T-cell-mediated autoimmune disease in general. Animals are scored according to the following clinical signs and duration of symptoms:

| | | Maximal | |
|---|---|---|---|
| EAE | | cumulative | |
| score | Clinical signs | duration | |
| 0 | Asymptomatic | End of the study | |
| 1 | Discrete behavioral disorder with stereotypia, | 20 | weeks |
| | ptosis, nystagmus, discrete paresis, rubbing. | | |
| 2 | Moderate behavioral disorders | 4 | weeks |
| | (shaking, oculomotor paralysis, paresis with | | |
| | compensation). | | |
| 3 | Walking disorders (ataxia, lameness) | 2 | weeks |
| | without social or feeding behavior impact. | | |
| 4 | Severe behavioral disorders (paralysis, | <18 | h |
| | paresis) leading to lack of self feeding. | | |
| 5 | Coma. | <6 | h |
| 6 | Moribund. | <1 | h |

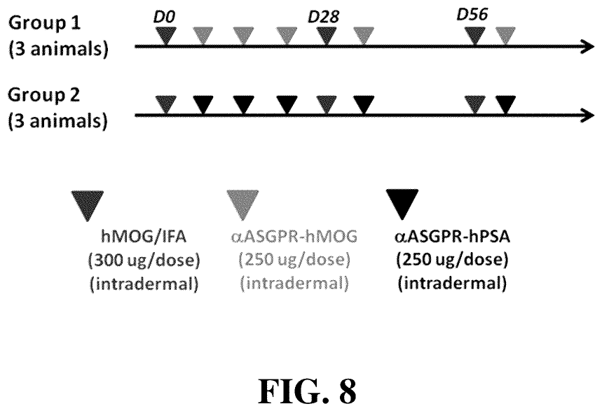

Experimental Design. The Experimental Design is summarized in FIG. 8. Group 1 (Experimental group: consisting of 3 cynomolgus macaques) and Group 2 (Control group: consisting of 3 cynomolgus macaques) were injected with hMOG in incomplete Freund's adjuvant on days 0, 28 and 56. Experimental Group 1 animals received anti-ASGPR-hMOG injections on days 7, 14, 21, 35, and 63. Control group 2 animals received anti-ASGPR-hPSA injections on days 7, 14, 21, 35, and 63.

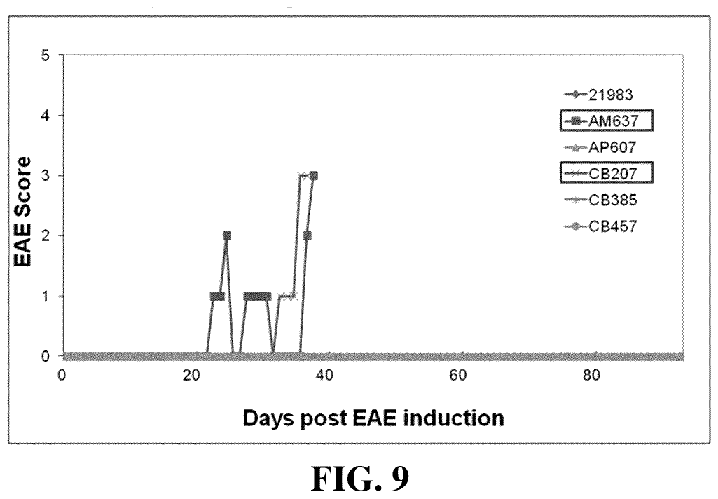

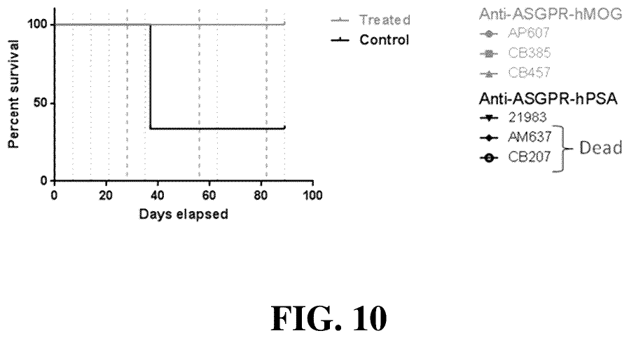

Anti-DC-ASGPR-MOG suppresses the development/progression of EAE in NHP. During the entire period of the experiment, animals were monitored daily to measure EAE score. None of the animals (ID numbers AP607, CB385, and CB457) treated with anti-DC-ASGPR-MOG showed any clinical signs of disease. These animals did not show escalated EAE disease scores. However, two (AM637 and CB207) out of three animals (AM637, CB207, and 21983) in the control group displayed escalated EAE scores between days 20 and 37 (when both animals died; FIGS. 9 and 10). Taken together, the inventors conclude that anti-DC-ASGPR-MOG is able to suppress the development/progression of EAE in NHP.

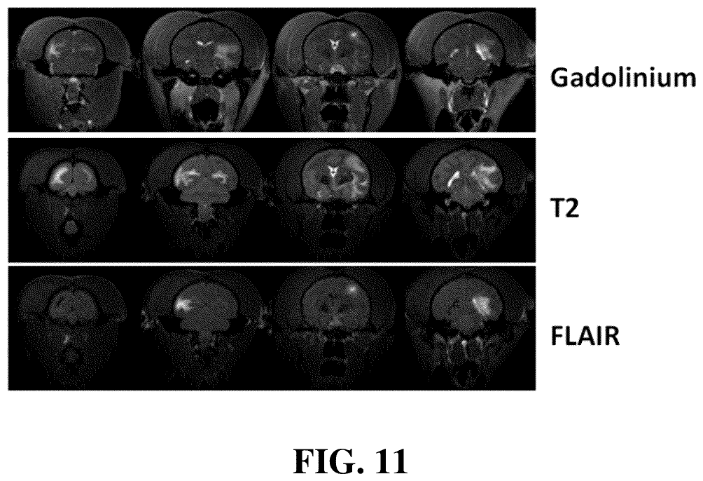

To further confirm whether the clinical signs observed (FIGS. 9 and 10) in the animals in the control group (Group 2 in FIG. 8) were due to inflammation in the brain, magnetic resonance imaging (MRI) of the brain of animal AM637 on day 22 was performed. FIG. 11 shows that i.v. administered gadolinium is dispersed, indicating that there was leakage in the blood vessels into the brain. A T2 image also shows the accumulation of water in several spots, indicating that myelin in this animal was damaged and thus less able to exclude water. Considering Flair data along with T2 and gadolinium data, it was concluded that this animal had severe inflammation in the brain, along with demyelination, which is a typical sign of EAE.

In summary, the inventors demonstrated that 1) cynomolgus macaques develop EAE by immunizing with MOG peptide and an adjuvant and 2) anti-DC-ASGPR-MOG, but not anti-DC-ASGPR-hPSA, suppressed the development/progression of EAE in cynomolgus macaques.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method of inducing immune tolerance to at least one myelin sheath protein in a patient comprising administering to the patient an effective amount of a composition comprising a dendritic cell targeting complex comprising a dendritic cell antibody, or targeting fragment thereof, attached to the at least one myelin sheath protein, or antigenic fragment thereof.

2. The method of claim 1, wherein at least one myelin sheath protein is myelin basic protein (MBP), myelin oligodendrocyte glycoprotein (MOG), proteolipid protein (PLP), or myelin associated glycoprotein (MAG).

3. The method of claim 2, wherein at least one myelin sheath protein is MBP.

4. The method of claim 2, wherein at least one myelin sheath protein is MOG.

5. The method of any of claims 1-4, wherein the dendritic cell antibody specifically binds asialoglycoprotein receptor (ASGPR).

6. The method of any of claims 1-5, wherein the composition comprises multiple dendritic cell targeting complexes.

7. The method of claim 6, wherein the multiple dendritic cell targeting complexes comprise different myelin sheath proteins or different antigenic fragments of one or more myelin sheath proteins.

8. The method of claim 6 or 7, wherein each myelin sheath protein or antigen fragment is separately attached to a dendritic cell antibody, or a targeting fragment thereof.

9. The method of any of claims 1-8, wherein the dendritic cell antibody is attached to the myelin sheath protein using a peptide linker.

10. The method of any of claims 1-9, wherein the composition further comprises at least one tolerogenic adjuvant.

11. The method of claim 10, wherein the tolerogenic adjuvant is attached to the dendritic cell targeting complex.

12. The method of claim 11, wherein the tolerogenic adjuvant is conjugated to the dendritic cell targeting complex.

13. The method of claim 11, wherein the tolerogenic adjuvant is fused to the dendritic cell antibody, or targeting fragment thereof, and/or to the at least one myelin sheath protein.

14. The method of any of claims 10-13, wherein the tolerogenic adjuvant is selected from IL-10, dexamethasone, FK506 (Tacrolimus), cholera toxin B subunit, Escherichia coli heat-labile enterotoxin B subunit, IFN-beta, glucocorticoids, vitamin D3, and vitamin D3 analogues.

15. The method of any of claims 1-14, wherein the dendritic cell antibody is attached to at least one myelin sheath protein through binding polypeptides.

16. The method of claim 15, wherein the binding polypeptides are dockerin and cohesin.

17. The method of any of claims 1-16, comprising more than one administration of the composition.

18. The method of any of claims 1-16, wherein the composition is administered orally, intravenously, subcutaneously, intradermally, intramuscularly, nasally, by injection, by inhalation, mucosally, and/or using a nebulizer.

19. The method of any of claims 1-18, wherein the subject exhibits one or more symptoms of a demyelinating disease.

20. The method of any of claims 1-19, wherein the subject has been diagnosed with a demyelinating disease.

21. The method of any of claims 1-20, wherein the subject is at risk for a demyelinating disease.

22. The method of any of claims 19-21, wherein the demyelinating disease affects the central nervous system.

23. The method of claim 22, wherein the demyelinating disease is an idiopathic inflammatory demyelinating disease.

24. The method of claim 22, wherein the demyelinating disease is multiple sclerosis, neuropathy, central pontine myelinolysis, tabes dorsalis, transverse myelitis, Devic's disease, progressive multifocal leukoencephalopathy, optic neuritis, or leukodystrophy.

25. The method of claim 24, wherein the demyelinating disease is multiple sclerosis.

26. The method of any of claims 19-21, wherein the demyelinating disease affects the peripheral nervous system.

27. The method of claim 26, wherein the demyelinating disease is Guillain-Barré syndrome, chronic inflammatory demyelinating polyneuropathy, anti-MAG peripheral neuropathy, Charcot-Marie-Tooth Disease, copper deficiency, or progressive inflammatory neuropathy.

28. The method of any of claims 1-24, further comprising preparing the composition.

29. The method of any of claims 1-28, further comprising measuring antibodies against the at least one myelin sheath protein in the subject after administering the composition.

30. A method for treating a demyelinating disease in a subject comprising administering to the subject a pharmaceutically acceptable vaccine composition comprising at least a first ASGPR antibody, or binding fragment thereof, attached to myelin basic protein (MBP) and/or myelin oligodendrocyte glycoprotein (MOG), or antigenic fragment thereof.

31. The method of claim 30, wherein the ASGPR antibody, or binding fragment thereof, is fused to MBP or MOG, or an antigenic fragment thereof.

32. The method of claim 30 or 31, wherein the subject is administered the vaccine composition multiple times.

33. The method of claim 32, wherein the composition is administered orally, intravenously, subcutaneously, intradermally, intramuscularly, nasally, by injection, by inhalation, muscosally, and/or using a nebulizer.

34. The method of any of claims 30-33, wherein the subject exhibits one or more symptoms of a demyelinating disease.

35. The method of any of claims 30-33, wherein the subject has been diagnosed with a demyelinating disease.

36. The method of any of claims 30-33, wherein the subject is at risk for a demyelinating disease.

37. The method of any of claims 34-36, wherein the demyelinating disease affects the central nervous system.

38. The method of claim 37, wherein the demyelinating disease is an idiopathic inflammatory demyelinating disease.

39. The method of claim 38, wherein the demyelinating disease is multiple sclerosis, neuropathy, central pontine myelinolysis, tabes dorsalis, transverse myelitis, Devic's disease, progressive multifocal leukoencephalopathy, optic neuritis, or leukodystrophy.

40. The method of claim 39, wherein the demyelinating disease is multiple sclerosis.

41. The method of any of claims 34-36, wherein the demyelinating disease affects the peripheral nervous system.

42. The method of claim 41, wherein the demyelinating disease is Guillain-Barré syndrome, chronic inflammatory demyelinating polyneuropathy, anti-MAG peripheral neuropathy. Charcot-Marie-Tooth Disease, copper deficiency, or progressive inflammatory neuropathy.

43. The method of any of claims 30-42, further comprising preparing the composition.

44. The method of any of claims 30-43, further comprising measuring antibodies against the at least one myelin sheath protein in the subject after administering the composition.

45. A composition comprising at least a first ASGPR antibody, or binding fragment thereof, attached to myelin basic protein (MBP) and/or myelin oligodendrocyte glycoprotein (MOG), or antigenic fragment thereof.

46. The composition of claim 45, wherein the dendritic cell antibody is attached to the myelin sheath protein or antigenic fragment thereof using a peptide linker.

47. The composition of any of claims 45-46, wherein the composition further comprises at least one tolerogenic adjuvant.

48. The composition of claim 47, wherein the tolerogenic adjuvant is attached to the dendritic cell targeting complex.

49. The composition of claim 48, wherein the tolerogenic adjuvant is conjugated to the dendritic cell targeting complex.

50. The composition of claim 48, wherein the tolerogenic adjuvant is fused to the dendritic cell antibody, or targeting fragment thereof, and/or to the at least one myelin sheath protein.

51. The composition of any of claims 47-50, wherein the tolerogenic adjuvant is selected from IL-10, dexamethasone, FK506 (Tacrolimus), cholera toxin B subunit, Escherichia coli heat-labile enterotoxin B subunit, IFN-beta, glucocorticoids, vitamin D3, and vitamin D3 analogues.

52. The composition of any of claims 45-51, wherein the dendritic cell antibody is attached to at least one myelin sheath protein or antigenic fragment thereof through binding polypeptides.

53. The composition of claim 52, wherein the binding polypeptides are dockerin and cohesin.
